SEQUENCE LISTING (44 sequences)

SEQ ID NO: 1 - DNA, 1476 bp, Amaranthus tuberculatus
atggaaatattatacacttatttagccatattttctactattttcttcctctataaaatcattcattcccttaaaccaaacaacaaaaaactcccaccaagcccaccatcatacccaatattgggccacttacaccttctaaaacccccattccaccgtacactccaatctctagcccaacgttacggtccaatcttctctctccaattgggccttcaacgggccttggtagtttcctccgcatgggccgccgaagaatgtttcagtcaaaacgacgtcgtttttgcaaacagaccaaaattcatagtaggccaacatttaggatacaaccactctatccttatctggtccccttatggggactactggcggaacctccgacgtgttacaaccattaccatgttatccatgagacggatcaacgaagcgggtccgacccggaaacttgagatccgaaacatgataatgggccttctagaaacctcaaaaggtgggagtgggacccagaaagtgaacatgaatgatgtacttagtaaacttgcaagaaattttgtaatgaggatagtaaatggaaaatcatgggaaaaaatgattataaaaccacctgaaaattttatgactatttgtgattttttgccatttttaagatgggtggattttaaagggatagaaaaggatatgaaggagaaacagattgaaagagatgagtttttacaaagtttggttgatgaaattagagaaagtagaaagaaaggtgaggatttagggatgagtactttgattgaacagttgctggatttgcaagaagctgaacctgatcaatacacagatgaaactatcaaaggaatcattttggtaatgttactagcgggatcagaaaccacagcacgaaccttagaatgggcattatcaaatctcattaaccacccagaaatccttgcgaaagcaagaacagaaatcgacctccatgtaggcaaggaacgactagtagatgattctgatctaccaaaattaacgtatacacgttgtattgtatacgaaactctaagactgtttccggctgcaccacttttagtacctcatttttcgtcacaagattgcaccataggagggtatcacgtaccaaaagggacaatgcttttcgtaaatgcttgggctatacacagagacccgaccctatgggatgagcctactgtgtttaagcccgagaggttcgaaaaggagaaagaagggtttaagtttatgccctttggaattggaaggagatcttgtccagggaataatgtggctcttaggaatgtttcattgacattggctactcttattcagtgctttgattgggaagcttccgagagtggatcaattgatctaaccgaaaagcgtggtgctggtgctatcattgtccctaaggaaaagccattagaggctatatgtcgacctcgaccttcaatggaggattttctcgttaaactttag

SEQ ID NO: 2 - protein, 491 aa, Amaranthus tuberculatus
MEILYTYLAIFSTIFFLYKIIHSLKPNNKKLPPSPPSYPILGHLHLLKPPFHRTLQSLAQRYGPIFSLQLGLQRALVVSSAWAAEECFSQNDVVFANRPKFIVGQHLGYNHSILIWSPYGDYWRNLRRVTTITMLSMRRINEAGPTRKLEIRNMIMGLLETSKGGSGTQKVNMNDVLSKLARNFVMRIVNGKSWEKMIIKPPENFMTICDFLPFLRWVDFKGIEKDMKEKQIERDEFLQSLVDEIRESRKKGEDLGMSTLIEQLLDLQEAEPDQYTDETIKGIILVMLLAGSETTARTLEWALSNLINHPEILAKARTEIDLHVGKERLVDDSDLPKLTYTRCIVYETLRLFPAAPLLVPHFSSQDCTIGGYHVPKGTMLFVNAWAIHRDPTLWDEPTVFKPERFEKEKEGFKFMPFGIGRRSCPGNNVALRNVSLTLATLIQCFDWEASESGSIDLTEKRGAGAIIVPKEKPLEAICRPRPSMEDFLVKL

SEQ ID NO: 3 - DNA, 1476 bp, Amaranthus tuberculatus
atggaaatattatacacttatttatccatatttgctactattttcttcctctataaaatcattcaatcccttaaaccaaacaacaaaaaactcccaccaagcccaccatcatacccaatattgggccacttacaccttctaaaacccccattccaccgtacactccaatctctagcccaacgttacggcccaatcttctctctccaattgggccttcaacgggccttggtagtttcctccgcatgggccgccgaggaatgtttcagtcaaaacgacgtcgtttttgcaaacagaccaaaattcataataggacaacatttaggatacaaccactctatccttatctggtccccttatggggactactggcggaacctccgacgtgttacaaccattaccatgttatccatgagacggatcaacgaagcgggtccgacccggaaacttgagatccgaaacatgataatggaccttctagaaacctcaaaaggtgggagtgggacccagaaagtgaatatgaatgatgttcttagtaaacttgcaagaaattttgtaatgaggatagtaaatggaaaatcatgggaaaaaatgattataaaaccccctgaaaatttgatgactatttgtgattttttgccatttttaagatgggtggattttaaagggatagagaaggatatgaaggagaaacagattgaaagagatgagtttttacaaagtttggttgatgaaattagagaaagtaggaagaaaggtgaggatttagggatgaatactttgattgaacagttgctggatttgcaagaagctgaacctgatcaatactctgatgaaactatcaaaggaatcattttggtaatgttactagcgggatcagaaaccacagcacgaaccttagaatgggcattatcaaatctcattaaccacccagaaatcctcgcgaaagcaagaacagaaatcgacctccatgtaggcaaggaacgactagtagatgattctgatctaccaaaattaacgtatacacgttgtattgtatacgaaactctaagactatttcctgctgcaccactattggtaccacatttttcgtcacaagattgcaccataggagggtatcacgtaccaaaagggacaatacttttcgtaaatgcttgggctatacatagagacccaaccttatgggacgagcctaccgtgtttaagcccgagaggttcgaaaaggagaaagaagggtttaagtttatgccctttggaattggaaggagatcttgtccggggaataacgtggctcttaggaatgtttcattgacattggctactcttattcagtgctttgattgggaaggggccgagagtggatcaattgatctaactgaaaagcgtggtgttggtgctatcattgtccctaaggaaaagccattagaggctatatgtcgacctcgaccttcaatggaggatttcctcgctaaaatttag

SEQ ID NO: 4 - protein, 491 aa, Amaranthus tuberculatus
MEILYTYLSIFATIFFLYKIIQSLKPNNKKLPPSPPSYPILGHLHLLKPPFHRTLQSLAQRYGPIFSLQLGLQRALVVSSAWAAEECFSQNDVVFANRPKFIIGQHLGYNHSILIWSPYGDYWRNLRRVTTITMLSMRRINEAGPTRKLEIRNMIMDLLETSKGGSGTQKVNMNDVLSKLARNFVMRIVNGKSWEKMIIKPPENLMTICDFLPFLRWVDFKGIEKDMKEKQIERDEFLQSLVDEIRESRKKGEDLGMNTLIEQLLDLQEAEPDQYSDETIKGIILVMLLAGSETTARTLEWALSNLINHPEILAKARTEIDLHVGKERLVDDSDLPKLTYTRCIVYETLRLFPAAPLLVPHFSSQDCTIGGYHVPKGTILFVNAWAIHRDPTLWDEPTVFKPERFEKEKEGFKFMPFGIGRRSCPGNNVALRNVSLTLATLIQCFDWEGAESGSIDLTEKRGVGAIIVPKEKPLEAICRPRPSMEDFLAKI

SEQ ID NO: 5 - DNA, 30 bp, Artificial Sequence
  synthetic
gtactttgattgaacagttgctggatttgc

SEQ ID NO: 6 - DNA, 24 bp, Artificial Sequence
  synthetic
aggttcgtgctgtggtttctgatc

SEQ ID NO: 7 - DNA, 27 bp, Artificial Sequence
  synthetic
tgtggagaagtaggctctggaaaatca

SEQ ID NO: 8 - DNA, 26 bp, Artificial Sequence
  synthetic
tccgaccataaacttcaacagtgcct

SEQ ID NO: 9 - DNA, 32 bp, Artificial Sequence
  synthetic
ggtgttacaatggatcatattggtgtcaaagc

SEQ ID NO: 10 - DNA, 31 bp, Artificial Sequence
  synthetic
gatgccttaatagttctgccattgtccattc

SEQ ID NO: 11 - DNA, 28 bp, Artificial Sequence
  synthetic
tcaaacccactcataaagaaggtcgcac

SEQ ID NO: 12 - DNA, 30 bp, Artificial Sequence
  synthetic
tcatctcattagaactttcccacattgctg

SEQ ID NO: 13 - DNA, 30 bp, Artificial Sequence
  synthetic
gatccatatcaattcaaggtgtcccacatg

SEQ ID NO: 14 - DNA, 29 bp, Artificial Sequence
  synthetic
ggaacaacatacacatgcgacaataccag

SEQ ID NO: 15 - DNA, 25 bp, Artificial Sequence
  synthetic
accgaaagcatttgtcgttgtctcg

SEQ ID NO: 16 - DNA, 26 bp, Artificial Sequence
  synthetic
tgggttggaggatttcagcaagaact

SEQ ID NO: 17 - DNA, 23 bp, Artificial Sequence
  synthetic
tggacgaagtggcagtggaaaga

SEQ ID NO: 18 - DNA, 25 bp, Artificial Sequence
  synthetic
acgcccatcctctccttatccttgt

SEQ ID NO: 19 - DNA, 26 bp, Artificial Sequence
  synthetic
accatcatcgtcggttgtgtttctct

SEQ ID NO: 20 - DNA, 25 bp, Artificial Sequence
  synthetic
tcctccaaccctttcgcaatctctt

SEQ ID NO: 21 - DNA, 24 bp, Artificial Sequence
  synthetic
gtggtgccaagaaggttgtcattt

SEQ ID NO: 22 - DNA, 20 bp, Artificial Sequence
  synthetic
agggagcaaggcagttggtg

SEQ ID NO: 23 - DNA, 29 bp, Artificial Sequence
  synthetic
cgtgtgattgaaagatttgagaaggaagc

SEQ ID NO: 24 - DNA, 21 bp, Artificial Sequence
  synthetic
ataccacgctcacgctctgct

SEQ ID NO: 25 - DNA, 24 bp, Artificial Sequence
  synthetic
cttgtgagaagaactggtagcaaa

SEQ ID NO: 26 - DNA, 28 bp, Artificial Sequence
  synthetic
gtacttaatcagcctagacaaagaaagg

SEQ ID NO: 27 - DNA, 46 bp, Artificial Sequence
  synthetic
gaaggtcggagtcaacggattccaatctctagcccaacgttacggt

SEQ ID NO: 28 - DNA, 46 bp, Artificial Sequence
  synthetic
gaaggtgaccaagttcatgctccaatctctagcccaacgttacggc

SEQ ID NO: 29 - DNA, 20 bp, Artificial Sequence
  synthetic
caacgggccttggtagtttc

SEQ ID NO: 30 - DNA, 51 bp, Artificial Sequence
  synthetic
gaaggtcggagtcaacggatttttgcaaacagaccaaaattcatagtaggc

SEQ ID NO: 31 - DNA, 51 bp, Artificial Sequence
  synthetic
gaaggtgaccaagttcatgcttttgcaaacagaccaaaattcataatagga

SEQ ID NO: 32 - DNA, 20 bp, Artificial Sequence
  synthetic
cttatggggactactggcgg

SEQ ID NO: 33 - protein, 496 aa, Spinacia oleracea
MEMEKIYTLLPLFAIIFFLYKTILPSNPKNKNLPPPSPPSIPILGHLHLLKPPFHRTLQSLSQRYGPIFSLRLGCQPVLVVSSPWAAEECFNQNDIVFANRPKFIIGEHLGYNHSLLIWSPYGEHWRNLRRVATLTMLSFRRINEAGPTRKMEIRNMISELLEGGTRKVNLNQVFGKLARNFVMRIMNGKPWEKMVMSPPANLMTVCDFLPVLRWVGFRGIEKELINMKKERDEFLQGLVDECRETRRRGLSCNDDEQNGKTNILIDKLLDLQEAEPEYYTDDFLKGFILVMLLAGSETTSRTLEWAMSNLINHSEILSKARAEIDHHVGEGRLVDDSDLPKLSYIRCIINETLRLFPPAPLLVPHYSSKDCTVGGFHVAKGTMLFVNAWAIHRDPNLWDEPTVFKPERFEKEIEGFKYMPFGIGRRTCPGNNLALRNVTLALATLIQCFNWEPTEAGLVDLTEKSGAGAIIVPKEKPLEAICSPRSSMEDVLAQI

SEQ ID NO: 34 - protein, 498 aa, Beta vulgaris
MEMEIIYTSLSFFAIIFFLYKIILSKPKNKNLPPSPPSLPILGHLHLLKAPFHRTLQSLSQRYGPIFFLRLGCQPVLVVTSSWAAEECFSKNDIVFANRPKFIIGEHLGYNHSLLIWSPYGDYWRNLRRVTTITMLSLRRINEAGPTRKIEIDNMISELLQGGTRKRVNLNAIFEKVARNFMMRVVNGKTWEKMIIRPPSNLMTICDFFPFLRWVGFNGIEKELRKLKKERDEFLQSLVDECRETRGGISCDEQIGENTNLIEQLLDLQQSEPEYYTDDVLKGIILVLILAGSETTARTLEWAMSNLINHPAVFAKARAEIDVNVGKGRLVEDCDLSKLSYIRCIIYETLRLFPPAPLLVPHYSSQDCNIGGYDVPKGTILFVNAWALHRDPNQWDDPTEFKPERFEKEKEGFKFMPFGIGRRACPGNNFALRNVSLTLATLIQCFDWEATEAGKVDLTEKCSAGAVIVPKEKPLEATCCPRSSMEDTFSHKFDNVSV

SEQ ID NO: 35 - protein, 490 aa, Chenopodium quinoa
MEMEKLYTILSLFAIIFFIYKIILTSKPKNKNLPPSPPSIPILGHLYLLKPPFHRTLLSLSERYGPIFSLQLGCQPVLVVSSPWAAEECFNQNDIVFANRPNFIVGEHLGYNHSLLIWSPYGDHWRNLRRVSALTMLSFRRINEAGPTRRSEIRNMISELVSSGTRKVNLYELFGKVSRNLVMRVVNGKPWERMIMKPPAELMKICDFLPVLKWVGFRGIEKELKNLMKERDSFMQSLVDEIRESKGGTNVDGKTTKCLIEQLLDLQQTDPDYYTDKTIKGIILVMLLAGSETTTRTLEWAMSNLINNPEVLAKARAEIDLHVGKGRLVDDSDLPKLSYIKCIVNETLRLFPPAPLLVPHCSSKDCTIGGFHVPKGTILFVNAWAIHRDPNLWDEPIVFKPERFENEIEGFKFMPFGIGRRACPGNNFALRNVNLLVATLIQCFDWEAAEDGLVDLTESHGAGAIIVPKDKHLEAICRPRSSMEEILGQS

SEQ ID NO: 36 - protein, 505 aa, Citrus clementina
MENTVILYSALLLLFLNIIIIALKFSYTRRKNLPPSPPSIPIIGHLHLIKQPMHRILQSLSQKYGPIISLRFGSRLVIVVSSSEAAEECFTKNDIVFANRPKFLTGKHLGYNYTVVTQASYGEHWRNLRRITAIEVFSSHRLSTFLPSRREEIKRLLKKLLSTGSRQGFSKVELKTALSELTFNIMMRMVAGKRYYGDDVEDEEEARRFRTIIKEAAAYGGATNAEDFLPILKWIDVGDHKKRILRFSRTTDAFLQGLIDEHRTKKPGSESTNTMIDHMLALQESQPEYYTDQIIKGLILVMLLAGTDTSAVTIEWAMSNLVNNPEVLEKARAELDSKVGQEYLIDEPDLSKLHYLQSVISETLRLYPAAPLLVPHQSSDDCTVGGYHVPRGAILLVNAWTIHRDPKLWNDPNNFRPERFEKGECEAHKLMPFGLGRRACPGSGLAQRVVGLTLGSLIQCFEWLRIDEEKVDMTEGRGITMPKAKPLEVMCRARPIVNNVSELFD

SEQ ID NO: 37 - protein, 498 aa, Glycine max
MTPFYFLLFAFILFLSINFLIQTRRFKNLPPGPFSFPIIGNLHQLKQPLHRTFHALSQKYGPIFSLWFGSRFVVVVSSPLAVQECFTKNDIVLANRPHFLTGKYIGYNNTTVAVSPYGDHWRNLRRIMALEVLSTHRINSFLENRRDEIMRLVQKLARDSRNGFTKVELKSRFSEMTFNTIMRMVSGKRYYGEDCDVSDVQEARQFREIIKELVTLGGANNPGDFLALLRWFDFDGLEKRLKRISKRTDAFLQGLIDQHRNGKHRANTMIDHLLAQQQSQPEYYTDQIIKGLALVMLLAGTDTSAVTLEWAMSNLLNHPEILKKAKNELDTHIGQDRLVDEPDIPKLPYLQSIVYETLRLHPAAPMLVPHLSSEDCTIGEYNIPQNTILLVNAWAIHRDPKLWSDPTHFKPERFENESEANKLLPFGLGRRACPGANLAQRTLSLTLALLIQCFEWKRTTKKEIDMTEGKGLTVSKKYPLEAMCQVCQSLTVKDIYNF

SEQ ID NO: 38 - protein, 484 aa, Solanum lycopersicum
MEIGFILPTILLIFSIFFFRKLQNARKKNLPPSPPFLPIIGHLHLLKSPIHQTFKSLSSKYGPIMYLHFGTSQVIIVSSASIAEQCFTKNDIIFANRPKSLASKHLGYNHTTIGFSPYGDHWRNLRRISNIQIFSTFTLNNSSSIRTEEVQFVVKKLAQEYKGGSTQKVKLKILFEKLVYDVLTKMVAGKRWAESSTDDLFGPTMIMNICDYIPILKWIRFQGLEKNLVELKIRRDEFLQGLIDECRKSRADKKTIIHTLLSLQRDQPECYTDDIIKGVIMVMFTAGTHTSAVTMEWAMSLLLNHPEVMKKARLEIDNLIGETRPLEEPDILKLPYLRCIINETLRLFPAGPLLVPHFSTQECTIEGYHIPKSTILFVNIWEIQRDSKIWEDANEFKPERFEGGIEGCKFIPFGMGRRACPGYGLAIRLIGLVLGLFIQCFEWERIGDELVSLDESCGLMLSKLEPLEALYRPRESMVALLSQL

SEQ ID NO: 39 - protein, 506 aa, Helianthus annuus
MEIPYLLTTTLLLLLFTTLYLLLRRRSSTLPPTIFPSLPIIGHLYLLKPPLYRTLAKLSAKHGPILRLQLGFRRVLIVSSPSAAEECFTRNDIVFANRPKMLFGKIIGVNYTSLAWSPYGDNWRNLRRIASIEILSIHRLNEFHDIRVEETRLLIQKLLSACNSGSSQVTMKFSFYELTLNVMMRMISGKRYFGGDNPELEEEGKRFRDMLDETFVLAGASNVGDYLPVLSWLGVKGLEKKLIKLQEKRDVFFQGLIDQLRKSKGTEDVNKRKTMIELLLSLQETEPEYYTDAMIRSFVLVLLAAGSDTSAGTMEWVMSLLLNHPQVLKKAQNEIDTVIGNNRLVDESDIPNLPYLRCIINETLRLYPAGPLLVPHEASSDCVVGGYNVPRGTMLIVNQWAIHHDPKVWDEPETFNPERFEGLEGTRDGFKLLPFGSGRRSCPGEGLAVRMLGMTLGSIIQCFDWERTSEELVDMTEGPGLTMPKAIPLVAKCKPRVEMTNLLSDL

SEQ ID NO: 40 - protein, 535 aa, Solanum tuberosum
MQHHRIKPFLPCALNLLIRRPKVVFTKLLFSLLNSVVKVLLSYGESYWFMNMEIGFTFSIILLIFSIFIFLKLQNARKKNLPPSPPSLPIIGHLHLLKSPIHQTFKSLSSKYGPIIYLHFGTSKVIIVSSASIAEQCFTKNDIIFANRPKSLASKHLGYNHTTIGFSPYGDHWRNLRRISNIQIFSTFTLNNSSSVRTEEVQFMAKKLTLDYKGGITQKVKLKILFEKLVYDVLTKMVAGKRWAEPSTDDLFGPTMIMNICDYIPVLKWVGFQGLEKNLVELKIRRDKFLQGLIDECRKSRADKKTIIHTLLSLQGDQPECYTDDIIKGVIMVMFTAGTHTSAVTMEWAMSLLLNHPEVMKKARLEIDNLIGETRPLEEPDILKLPYLRCIINETLRLFPAGPLLVPHFSTQDCTIEGYHIPKGTILFVNIWEIQRDRKIWEEANEFKPERFVGGIEGCKFIPFGMGRRACPGSGLAMRLIGLVLGLFIQCFEWERIGDELVGLDESCGLMLSKLEPLEALYRPRESMVTLLSQL

SEQ ID NO: 41 - protein, 499 aa, Prunus persica
MEDILFYTSLTLIFILFTFKFLVQPNRRRYKNLPPTPPSLPILGHLHLLKPPVHRTFHRLSQKYGAVFSLWFGSHRVVIVSSPSAVEECFTKNDIVLANRPRLLFGKHLAYNYTTVVAAPYGDHWRNLRRIGTTEIFSTARLQTFSEIRKDEVKHLLLKLSQNARDGFAKVELKSMFNELTFNIIMTMVAGKRYYGDDVSVDKEEAKQFRQIMSDVFFYGGAANPADFLPILNWVGRGGYEKKVKTLAKRTDEFLQALIDEHKSKGKNGTTMIDHLLSLQESQPEYYTNQIIKGLILVMLLAGTDTSAVTLEWAMSNLLNNPHVLKKARVELDAQLGEENLVDEPDLSKLPYLQNIISETLRLCPAAPLLVPHFSSDDCTIGGFDVPRDTMILINAWALHRDPQLWDDPESFMPERFESGGDLSHKLIPFGLGRRACPGLGLAQRVVGLTLGSLIQCFEWERITKEEIDMAEGKGLTMPKVVPLEAMCRARSVMTKVLS

SEQ ID NO: 42 - protein, 500 aa, Gossypium hirsutum
MEESITLYSFLSFIFFIICLNLFLRSRRHRKNLPPTPPSLPIVGHLHLLKPPIHRLHHTFSQKYGPIFSVKLGSRLMVVVSSSTAAEECLIKNDIIFANRPKFIIAKHLGYNYTTLISSSYGDHWRNLRRIGATEIFSSGRLNASVNVRKDETRRLMLRLSTDSRQDFVKVELKPMLSDLTFNNIMRMLAGKRYYGDEVTNEEEAREFRELMVEVAKNSGTGNPADYLPVLNWFGLGFEGKLKKLGKRLDGFLQKLVDDHRSNKLKNNSMIDHLLSMQESDPLYYTDEIIKGFIMVILFAGTDTSSVTMEWAMANLLNHPQVLKKARDEIDNLIGEEKLIEESDVPKLHYLQSIIYETLRLYPAAPLLVPHMPSTDCSIGGYDVPSGTIVLVNAWAIHRDPNVWDDPTSFKPERFDGNSEKIEHSQKLLPFGLGRRSCPGANLAQRTVGLALGSLIQCFEWERIEGKEIDMSEGKGTIMPKLHPLEALCKARPIVDKLFY

SEQ ID NO: 43 - protein, 500 aa, Manihot esculenta
MEDTLLYLFLFILFFLALKVFQSRIRRQNLPPSPPAIPIIGHLNLLKPPMHRTFHSLAEKYGPIIFLRFSCRPVVIVSSSSAAEECFTKNDIVFANRPKLLTGKHIAYNYTTLLHAPYGDHWRNLRRIGSIEIFSTHRLNVLQSIRKDEIKRLLTKLSYQSLRDFAKVELKSVFNELTFNIMMRMIAGKRYYGDDVSNEKEARKFREMMKEIITYSGVSNPGDFMPILNWISERKVIMLAKKVDKFLQGLIDEHRNNKENLERKHTMIDHLLALQESQPDYYTDEIIKGLIQTMLFAGTDTSAVTLEWAMSNLLNQPSILRKARDEIETQVGQECLLDESHLPKLPYLQNIVSETLRLYPAAPLLVPHMSSDDCTVGGYDIPRGTLLLVNAWAIHRDPTLWDDPTSFRPERYGGGEEDVHYKLMPFGLGRRSCPGSGLAQRVVGLTLGSLVQCFEWERVSDEEIDMREGRGITMPKAEPLEAMCKVRPFAEKILPLAQPI

SEQ ID NO: 44 - protein, 501 aa, Sinapis alba
MEEAQTLTLTLLFIVLTIIFFIRRHRITRKLKLPPTPPFALPVIGHLRLLKPPLHRVFLSISQSLGGAPIFSLRLGSQLVFVVSSHSIAEECFTKNDVVLANRPNTIASKHVSYDHTTMVSAPYGEHWRNLRRIGAVEIFSAHRLNRFLSIRQDEIRRLIVRLAMNSSHEIAKVEINSMFSDLTFNNIMRMVAGKRYYGDASEESSEAKHVRQLIADLTSVFGAGNAADYLPFLRWVTGFEKRVKELAGRFDEFLQGLVDERRAAKKKGNTMIDHLLSLQETQPEYYTDRTIKGTILSLILAGTDTSAVTLEWALSSLLNHPEELRKAREEIDSEIGLDRFVEESDISNLPYLQNIVSETLRLFPAGPLMVPHVASEDCKVGGYDMPRGTTLLVNLWAIHRDPQLWDDPEIFKPERFEKEGVAHKLMTFGLGRRACPGSGLAQRLVSLTLASLIQCFEWERIGEEEVDMTEAGGVTMRKARPLVAMCRARTFVGKILHKSA

(19) United States
(12) Patent Application Publication
(10) Pub. No.: US 2022/0372506 A1
(43) Pub. Date: Nov. 24, 2022

(54) CYP81E GENES CONFERRING HERBICIDE TOLERANCE

(71) Applicants: Colorado State University Research Foundation, Fort Collins, CO (US); THE BOARD OF TRUSTEES OF THE UNIVERSITY OF ILLINOIS, Urbana, IL (US)

(72) Inventors: Todd Gaines, Fort Collins, CO (US); Marcelo Rodrigues Alves de Figueiredo, Fort Collins, CO (US); Patrick John Tranel, St. Joseph, IL (US); Darci Ann Giacomini, Lakewood, CO (US)

(21) Appl. No.: 17/755,163

(22) PCT Filed: Sep. 1, 2021
(86) PCT No.: PCT/US2021/048623

§ 371 Date: Apr. 22, 2022

Related U.S. Application Data
(60) Provisional application No. 63/073,276, filed on Sep. 1, 2020.

(51) Int. Cl. C12N 15/82; C12Q 1/6895; C12N 9/02

(52) U.S. Cl. CPC C12N 15/8274; C12N 15/8205; C12Q 1/6895; C12N 9/0071; C12Q 2600/13; C12Q 2600/158

(57) ABSTRACT

The present disclosure relates to a plant or plant part comprising a polynucleotide encoding a CYP81E polypeptide, the expression of the polynucleotide confers to the plant or plant part tolerance to synthetic auxin herbicides, such as 2,4-D. The disclosure further provides kits for identifying herbicide resistant plants and methods for determining whether a plant is herbicide resistant.

### CROSS REFERENCE TO RELATED APPLICATIONS

This application claims priority to provisional application U.S. Ser. No. 63/073,276, filed Sep. 1, 2020, which is incorporated herein by reference in its entirety.

### SEQUENCE LISTING

The instant application contains a Sequence Listing which has been submitted in ASCII format via Electronic Submission and is hereby incorporated by reference in its entirety. Said ASCII copy, created on Aug. 26, 2021, is named P13673WO00_ST25.txt and is 69,987 bytes in size.

### TECHNICAL FIELD

The present disclosure relates in general to compositions and methods for conferring plants with tolerance to herbicides.

### BACKGROUND

Weeds left uncontrolled can decrease the yields of several major crops by more than 50% in present North American agronomic systems. Many growers in the United States currently rely heavily on chemical means (i.e. herbicides) to control their weed populations, but the effectiveness of this approach is steadily declining due to growing numbers of herbicide-resistant weeds. While herbicide resistance has been present in the United States since the late 1950s, the widespread adoption of herbicide-tolerant crop varieties in the mid-1990s and overreliance on one or two herbicidal modes of action contributed to an exponential increase in the number of resistant weed species over the last two decades. There are currently 164 weed species in the United States with documented resistance to herbicides spanning one or more modes of action.

Understanding how weeds deal with herbicidal compounds to avoid damage is a major goal of weed science, both to generate workarounds to combat herbicide resistance and to gain insights into plant evolution. Research on herbicide-resistance mechanisms over the last several decades has largely been focused on mutations occurring within genes that encode the target enzymes that are directly inhibited by herbicides (target-site resistance). Only recently has significant progress been made on non-target-site-based resistance (NTSR) mechanisms, largely due to the increased availability of high-throughput whole genome/transcriptome analyses. This work has largely pointed to enhanced herbicide metabolism as a primary route of NTSR, but resistance mechanisms including reduced translocation and vacuolar sequestration have also been reported. Widespread use of herbicides to control weeds provides an excellent platform for studying rapid adaptation of plants to strong selection, and to address evolutionary questions that are increasingly tractable due to genomics advances.

Amaranthus tuberculatus is a highly problematic weed species for growers across the midwestern United States, due to both its high fecundity and ability to readily evolve resistance to herbicides. Since the report of ALS (acetolactate synthase)-inhibitor resistance in A. tuberculatus in 1993, this species has accrued resistances to herbicides spanning six additional sites of action. In 2016, a population was discovered in Illinois that carried five-way resistance, including resistance to photosystem II inhibitors, PPO (protoporphyrinogen oxidase) inhibitors, HPPD (4-hydroxyphenylpyruvate dioxygenase) inhibitors, and synthetic auxins. Two of the resistance traits (ALS and PPO) were found to be attributable to target site mutations, but both the HPPD-inhibitor- and synthetic auxin-resistance mechanisms were unknown. In 2012, a population was reported from Nebraska that was highly resistant to 2,4-D and was subsequently determined to be resistant to HPPD-inhibiting herbicides as well.

Herbicide tolerant plants are useful in systems in which a plurality of such plants are planted, and can produce a crop, and either prior to planting, or after planting, an herbicide is applied that would otherwise kill or harm the plants but for their tolerance to the herbicide. Undesirable plants are killed or damaged, and the tolerant plants survive. There is a need to produce such plants.

### SUMMARY

Compositions and methods for conferring herbicide tolerance to plants, plant parts, and plant cells are provided. Modified plants having tolerance to an herbicide, the modified plant comprising increased expression of a polynucleotide encoding a cytochrome P450 81E (CYP81E) polypeptide relative to an unmodified plant are provided. In certain embodiments, the modified plants comprise a heterologous polynucleotide encoding the CYP81E polypeptide. Progeny, plant parts, and plant cells of the modified plants are also provided.

Nucleic acid molecules capable of conferring herbicide tolerance comprising a nucleotide sequence selected from: (a) a nucleotide sequence encoding a CYP81E polypeptide, wherein the nucleotide sequence has at least 80%, at least 90%, at least 95%, at least 98%, or at least 99% sequence identity to SEQ ID NO: 1; or (b) a nucleotide sequence encoding a CYP81E polypeptide, wherein the CYP81E polypeptide has at least 80%, at least 90%, at least 95%, at least 98%, or at least 99% sequence identity to SEQ ID NO: 2 are provided.

Expression cassettes, vectors, biological samples, plants, plant parts, and plant cells comprising the aforementioned nucleic acid molecules are also provided.

CYP81E polypeptides comprising an amino acid sequence having at least 80%, at least 90%, at least 95%, at least 98%, or at least 99% sequence identity to SEQ ID NO: 2 are provided.

Methods for producing a plant with herbicide tolerance comprising increasing expression of a polynucleotide encoding a CYP81E polypeptide in the plant, wherein the herbicide tolerance of the plant is increased when compared to a plant that lacks the increased expression are provided. In certain embodiments, the methods comprise introducing to a plant cell a polynucleotide encoding the CYP81E polypeptide, wherein the polynucleotide is operably linked to a heterologous promoter functional in a plant cell; and regenerating a plant from the plant cell.

Methods for controlling undesired vegetation at a plant cultivation site comprising providing at the site a plant that comprises a polynucleotide encoding a CYP81E polypeptide, wherein expression of the polynucleotide confers to the plant tolerance to an herbicide; and applying to the site an effective amount of the herbicide are provided.

Methods for controlling the growth of an herbicide resistant weed at a plant cultivation site comprising contacting the weed with a composition comprising a polynucleotide that reduces expression or activity of a CYP81E polypeptide; and applying to the site an effective amount of the herbicide are provided.

Products prepared from the aforementioned plants, plant parts, and plant cells, wherein the product comprises the polynucleotide encoding the CYP81E polypeptide are provided. Methods for producing a plant product comprising processing the aforementioned plants or plant parts to obtain the plant product, wherein the plant product comprises the polynucleotide encoding the CYP81E polypeptide are also provided.

Methods for identifying an herbicide-resistant plant comprising providing a biological sample from a plant suspected of having herbicide resistance; quantifying expression of a CYP81E gene in the biological sample, wherein the CYP81E gene is differentially expressed in an herbicide-resistant plant compared to an herbicide-sensitive plant of the same species; and determining that the plant is herbicide-resistant based on the quantification are provided.

Kits for identifying an herbicide-resistant plant comprising at least two primers, wherein the at least two primers recognize a CYP81E gene that is differentially expressed in an herbicide-resistant plant compared to an herbicide-sensitive plant of the same species are also provided.

While multiple embodiments are disclosed, still other embodiments of the inventions will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE FIGURES

The following drawings form part of the specification and are included to further demonstrate certain embodiments or various aspects of the invention. In some instances, embodiments of the invention can be best understood by referring to the accompanying figures in combination with the detailed description presented herein. The description and accompanying figures may highlight a certain specific example, or a certain aspect of the invention. However, one skilled in the art will understand that portions of the example or aspect may be used in combination with other examples or aspects of the invention.

FIG. 1 is a schematic of the experimental design. Within each F2 population, plants were cloned and sprayed with high and low rates of tembotrione or 2,4-D. Based on their response, each plant was grouped into one of four categories: RR, resistant to both 2,4-D and tembotrione; RS, resistant to 2,4-D and sensitive to tembotrione; SR, sensitive to 2,4-D and resistant to tembotrione; and SS, sensitive to both 2,4-D and tembotrione. The four most resistant/sensitive plants from each category were chosen for RNA-seq analysis. This allowed for an N=8 comparison between resistant and sensitive plants for each herbicide using only 16 plants for each population.

FIG. 2A-B shows sliding window graphs of significantly differentially expressed genes and significant SNPs. FIG. 2A shows significantly differentially expressed genes (DEGs) between 2,4-D resistant and sensitive plants in CHR and NEB mapped on the A. hypochondriacus genome. Only genes with an FDR of 0.05 or less were considered significant. FIG. 2B shows single nucleotide polymorphisms (SNPs) that were statistically different between 2,4-D resistant and sensitive plants in CHR and NEB mapped on the A. hypochondriacus genome. Statistically significant SNPs were called if PLINK analysis returned a corrected p-value of 0.05 or less.

FIG. 3A-B shows allele-specific expression of all SNPs in the scaffold 4 hotspot region for the NEB population (FIG. 3A) and the CHR population (FIG. 3B). The location of each SNP is given across the x-axis and the results of a t-test for differential expression between the R and S allele (Benjamini and Hochberg adjusted P-value) is given above the bars for each locus.

FIG. 4 shows a phylogenetic tree of cytochrome P450 81E8 in an arbitrary subset of A. tuberculatus populations from Illinois, Nebraska, Missouri, and Canada. Samples from this study are indicated with their population name (“CHR” or “NEB”) as well as their 2,4-D phenotypic response. Samples beginning with a number or “N3” originated from Ontario and samples beginning with “B”, “F”, “J”, or “K” originated from Illinois and Missouri.

### DETAILED DESCRIPTION

Amaranthus tuberculatus has evolved resistance to 2,4-dichlorophenoxyacetic acid (2,4-D) and 4-hydroxyphenylpyruvate dioxygenase (HPPD) inhibitors in multiple states across the midwestern US. Two populations resistant to both mode-of-action groups, one from Nebraska (NEB) and one from Illinois (CHR), were studied using an RNA-seq approach on F2 mapping populations to identify the genes responsible for resistance.

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The term “about,” as used herein, refers to variation in the numerical quantity that can occur, for example, through typical measuring techniques and equipment, with respect to any quantifiable variable, including, but not limited to, mass, volume, time, and temperature. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

As used herein, the term “confer” refers to providing a characteristic or trait, such as herbicide tolerance or resistance and/or other desirable traits to a plant.

The term “control of undesired vegetation or weeds” is to be understood as meaning the killing of weeds and/or otherwise retarding or inhibiting the normal growth of the weeds. Weeds, in the broadest sense, are understood as meaning all those plants which grow in locations where they are undesired. The weeds of the present disclosure include, for example, dicotyledonous and monocotyledonous weeds. Dicotyledonous weeds include, but are not limited to, weeds of the genera: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and Apera. In addition, the weeds of the present disclosure can include, for example, crop plants that are growing in an undesired location. For example, a volunteer maize plant that is in a field that predominantly comprises soybean plants can be considered a weed, if the maize plant is undesired in the field of soybean plants.

As used herein, the term “DNA” or “DNA molecule” refers to a double-stranded DNA molecule of genomic or synthetic origin, i.e. a polymer of deoxyribonucleotide bases or a polynucleotide molecule, read from the 5′ (upstream) end to the 3′ (downstream) end. As used herein, the term “DNA sequence” refers to the nucleotide sequence of a DNA molecule. The nomenclature used herein corresponds to that of by Title 37 of the United States Code of Federal Regulations § 1.822, and set forth in the tables in WIPO Standard ST.25 (1998), Appendix 2, Tables 1 and 3.

As used herein, an “endogenous gene” or a “native copy” of a gene refers to a gene that originates from within a given organism, cell, tissue, genome, or chromosome. An “endogenous gene” or a “native copy” of a gene is a gene that was not previously modified by human action. Similarly, an “endogenous protein” refers to a protein encoded by an endogenous gene.

Generally, the term “herbicide” is used herein to mean an active ingredient that kills, controls or otherwise adversely modifies the growth of plants. The preferred amount or concentration of the herbicide is an “effective amount” or “effective concentration.” By “effective amount” and “effective concentration” is intended an amount and concentration, respectively, that is sufficient to kill or inhibit the growth of a similar, wild-type, plant, plant tissue, plant cell, or host cell, but that said amount does not kill or inhibit as severely the growth of the herbicide-resistant plants, plant tissues, plant cells, and host cells of the present disclosure. Typically, the effective amount of an herbicide is an amount that is routinely used in agricultural production systems to kill weeds of interest. Such an amount is known to those of ordinary skill in the art. Herbicidal activity is exhibited by herbicides useful for the present disclosure when they are applied directly to the plant or to the locus of the plant at any stage of growth or before planting or emergence. The effect observed depends upon the plant species to be controlled, the stage of growth of the plant, the application parameters of dilution and spray drop size, the particle size of solid components, the environmental conditions at the time of use, the specific compound employed, the specific adjuvants and carriers employed, the soil type, and the like, as well as the amount of chemical applied. These and other factors can be adjusted as is known in the art to promote non-selective or selective herbicidal action. Generally, the herbicide treatments can be applied PPI (Pre Plant Incorporated), PPSA (Post plant surface applied), PRE- or POST-emergent. Postemergent treatment typically occurs to relatively immature undesirable vegetation to achieve the maximum control of weeds.

By a “herbicide-tolerant” or “herbicide-resistant” plant, it is intended that a plant that is tolerant or resistant to at least one herbicide at a level that would normally kill, or inhibit the growth of, a normal or wildtype plant. Levels of herbicide that normally inhibit growth of a non-tolerant plant are known and readily determined by those skilled in the art. Examples include the amounts recommended by manufacturers for application. The maximum rate is an example of an amount of herbicide that would normally inhibit growth of a non-tolerant plant. For the present disclosure, the terms “herbicide-tolerant” and “herbicide-resistant” are used interchangeably and are intended to have an equivalent meaning and an equivalent scope. Similarly, the terms “herbicide-tolerance” and “herbicide-resistance” are used interchangeably and are intended to have an equivalent meaning and an equivalent scope. Similarly, the terms “tolerant” and “resistant” are used interchangeably and are intended to have an equivalent meaning and an equivalent scope. As used herein, in regard to an herbicidal composition useful in various embodiments hereof, terms such as herbicides, and the like, refer to those agronomically acceptable herbicide active ingredients (A.I.) recognized in the art. As used herein, an “herbicide tolerance trait” is a transgenic trait imparting improved herbicide tolerance to a plant as compared to the wild-type plant.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

As used herein, the term “isolated DNA molecule” refers to a DNA molecule at least partially separated from other molecules normally associated with it in its native or natural state. In one embodiment, the term “isolated” refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

As used herein, “modified”, in the context of plants, seeds, plant components, plant cells, and plant genomes, refers to a state containing changes or variations from their natural or native state. For instance, a “native transcript” of a gene refers to an RNA transcript that is generated from an unmodified gene. Typically, a native transcript is a sense transcript. Modified plants or seeds contain molecular changes in their genetic materials, including either genetic or epigenetic modifications. Typically, modified plants or seeds, or a parental or progenitor line thereof, have been subjected to mutagenesis, genome editing (e.g., without being limiting, via methods using site-specific nucleases), genetic transformation (e.g., without being limiting, via methods of Agrobacterium transformation or microprojectile bombardment), or a combination thereof. In one aspect, a modified plant provided herein comprises no non-plant genetic material or sequences. In yet another aspect, a modified plant provided herein comprises no interspecies genetic material or sequences.

As used herein, “plant” refers to a whole plant, any part thereof, or a cell or tissue culture derived from a plant, comprising any of: whole plants, plant components or organs (e.g., leaves, stems, roots, etc.), plant tissues, seeds, plant cells, and/or progeny of the same. A progeny plant can be from any filial generation, e.g., F1, F2, F3, F4, F5, F6, F7, etc. A plant cell is a biological cell of a plant, taken from a plant or derived through culture from a cell taken from a plant.

The term “polynucleotide” as used herein is a nucleic acid molecule comprising a plurality of polymerized nucleotides, e.g., at least about five consecutive polymerized nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single-stranded or double-stranded DNA or RNA. The polynucleotide optionally comprises modified bases or a modified backbone.

The polynucleotide can be, e.g., genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, amplicon, primer, oligomer, element, target, and probe and in some embodiments is single-stranded.

The term “primer” as used herein encompasses any nucleic acid that is capable of priming the synthesis of a nascent nucleic acid in a template-dependent process, such as PCR. Typically, primers are oligonucleotides from 10 to 30 nucleotides in length, but longer sequences may be used. Primers may be provided in single or double-stranded form. Probes may be used as primers, but are designed to bind to the target DNA or RNA and need not be used in an amplification process.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such as Agrobacterium or Rhizobium. Examples of promoters under developmental control include promoters that preferentially initiate transcription in certain tissues, such as leaves, roots, or seeds. Such promoters are referred to as “tissue preferred”. Promoters that initiate transcription only in certain tissue are referred to as “tissue specific”. A “cell type” specific promoter primarily drives expression in certain cell types in one or more organs, for example, vascular cells in roots or leaves. An “inducible” or “repressible” promoter is a promoter that is under environmental control. Examples of environmental conditions that may affect transcription by inducible promoters include anaerobic conditions or the presence of light. Tissue specific, tissue preferred, cell type specific, and inducible promoters constitute the class of “non-constitutive” promoters. A “constitutive” promoter is a promoter that is active under most environmental conditions.

As used herein, “recombinant,” when referring to nucleic acid or polypeptide, indicates that such material has been altered as a result of human application of a recombinant technique, such as by polynucleotide restriction and ligation, by polynucleotide overlap-extension, or by genomic insertion or transformation. A gene sequence open reading frame is recombinant if that nucleotide sequence has been removed from its natural context and cloned into any type of artificial nucleic acid vector. The term recombinant also can refer to an organism having a recombinant material, e.g., a plant that comprises a recombinant nucleic acid can be considered a recombinant plant.

“Regulatory elements” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory elements may include, but are not limited to, promoters, translation leader sequences, introns, and polyadenylation recognition sequences. Regulatory elements present on a recombinant DNA construct that is introduced into a cell can be endogenous to the cell, or they can be heterologous with respect to the cell. The terms “regulatory element” and “regulatory sequence” are used interchangeably herein.

A “sequence” means a sequential arrangement of nucleotides or amino acids. The boundaries of a protein-coding sequence may be determined by a translation start codon at the 5′-terminus and a translation stop codon at the 3′-terminus. In some embodiments, a protein-coding molecule may comprise a DNA sequence encoding a protein sequence. In some embodiments, a protein-coding molecule may comprise a RNA sequence encoding a protein sequence. As used herein, “transgene expression”, “expressing a transgene”, “protein expression”, and “expressing a protein” mean the production of a protein through the process of transcribing a DNA molecule into messenger RNA (mRNA) and translating the mRNA into polypeptide chains, which are ultimately folded into proteins.

As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, Calif.), MEGAlign (DNAStar Inc., Madison, Wis.), and MUSCLE (version 3.6) (Edgar, “MUSCLE: multiple sequence alignment with high accuracy and high throughput” Nucleic Acids Research 32(5):1792-7 (2004)) for instance with default parameters. An “identity fraction” for aligned segments of a test sequence and a reference sequence is the number of identical components that are shared by the two aligned sequences divided by the total number of components in the portion of the reference sequence segment being aligned, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence.

As used herein, “synthetic auxin herbicide” or “auxin herbicide” means any herbicide that exerts herbicidal activity through mimicking an endogenous plant auxin or inhibit the movement of auxinic compounds out of cells. Examples of synthetic auxin herbicides include benzoic acids, phenoxycarboxylic acids, pyridine carboxylic acids, quinoline carboxylic acids, semi-carbasones, Diflufenzopyr, 2,4-D, 2,4-DB, MCPA, MCPB, Mecoprop, Dicamba, Clopyralid, Fluroxypyr, Picloram, Triclopyr, Aminopyralid, Aminocyclopyrachlor, and Quinclorac.

As used herein, “vector” includes reference to a nucleic acid used in transfection of a host cell and into which can be inserted a polynucleotide. Vectors are often replicons. Expression vectors permit transcription of a nucleic acid inserted therein.

### CYP81E Polynucleotides

The plant hormone auxin serves as a central regulator of genes involved numerous plant growth, developmental, and response pathways. The naturally occurring active auxin is indole-3-acetic acid (IAA), but many other compounds have been found to mimic the function of IAA when applied to plants. This has led to the identification and commercialization of a number of compounds that function as effective herbicides. While corn and other monocotyledonous crops are naturally tolerant to low levels of synthetic auxin herbicides, dicotyledonous crops such as soybean and cotton are highly sensitive. Efforts to develop auxin herbicide tolerant varieties have been focused on the heterologous expression of enzymes that inactivate the auxin herbicide, thereby rendering otherwise sensitive plants tolerant to the herbicide.

Cytochrome P450 81E (CPY81E) sequences are provided that confer herbicide tolerance. Such sequences include the amino acid sequence set forth in SEQ ID NO: 2, and variants thereof. Also provided are polynucleotide sequences encoding such amino acid sequences, including SEQ ID NO: 1.

According to several embodiments crop plants are transformed with a gene encoding a CPY81E polypeptide capable of inactivating certain auxin herbicides and also, optionally, other types of herbicides.

Additional polynucleotide sequences encoding a CPY81E polypeptide may be identified using methods well known in the art based on their ability to confer tolerance to an herbicide of interest. For example, candidate CPY81E genes are transformed into and expressed in suitable yeast strains and selected on the basis of their ability to oxidize test herbicides in vitro (cf Siminszky et al (1999) PNAS (USA) 96:1750-1755). Suitable yeast strains include such as WAT11 or WAT21 which also comprise a suitable plant cytochrome P450 competent reductase. Following induction for a suitable period (for example, depending on the inducible promoter used in the transformation vector, with galactose) cells are grown up, harvested, broken, the microsome fraction prepared by the usual means and assayed with NADPH for the ability to oxidize 14C-labeled herbicide. Optionally, assays are carried out using whole cells in culture.

Alternatively, candidate CPY81E genes are expressed in tobacco, Arabidopsis, or other easily transformed, herbicide sensitive plant and the resultant transformant plants assessed for their tolerance to auxin herbicide(s) or other herbicides of interest. Optionally the plants, or tissue samples taken from plants, are treated with herbicide and assayed in order to assess the rate of metabolic conversion of parent herbicide to oxidized metabolic degradation products.

Those skilled in the art may also find further candidate CPY81E genes based on genome synteny and sequence similarity. In one embodiment, additional gene candidates can be obtained by hybridization or PCR using sequences based on the CPY81E nucleotide sequences noted above.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art. See, for example, Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

By “hybridizing to” or “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2×(or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone nucleotide sequences that are homologues of reference nucleotide sequences: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

Several embodiments also relate to the use of CYP81E or variants thereof that confer tolerance to herbicides, including auxin herbicides. “Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode CYP81E polypeptides described above. Naturally occurring allelic variants can be identified with the use of well-known molecular biology techniques, as, for example, with polymerase chain reaction (PCR) and hybridization techniques as outlined above. Variant polynucleotides also include synthetically derived polynucleotides, such as those generated, for example, by using site-directed mutagenesis but which still encode a CYP81E polypeptide conferring herbicide tolerance. Generally, variants of a particular polynucleotide will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide.

Variants of a particular polynucleotide encoding a CYP81E that confers herbicide tolerance are encompassed and can be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and algorithms described below. Where any given pair of polynucleotides is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

Methods of alignment of sequences for comparison are well known in the art and can be accomplished using mathematical algorithms such as the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; and the algorithm of Karlin and Altschul (1990) Proc. Natl. Acad. Sci. USA 872264, modified as in Karlin and Altschul (1993) Proc. Natl. Acad. Sci. USA 90:5873-5877. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA).

Several embodiments relate to increasing expression of a CYP81E gene in a plant. The term “increased expression” or “overexpression” as used herein means any form of expression that is additional to the original wild-type expression level. The original wild-type expression level might also be zero (absence of expression). Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the protein of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a CYP81E gene so as to control the expression of the gene.

Targeted modification of plant genomes through the use of genome editing methods can be used to increase expression of a CYP81E gene through modification of plant genomic DNA. Genome editing methods can enable targeted insertion of one or more nucleic acids of interest into a plant genome. Examples methods for introducing donor polynucleotides into a plant genome or modifying genomic DNA of a plant include the use of sequence specific nucleases, such as zinc-finger nucleases, engineered or native meganucleases, TALE-endonucleases, or an RNA-guided endonucleases (for example, a Clustered Regularly Interspersed Short Palindromic Repeat (CRISPR)/Cas9 system, a CRISPR/Cpf1 system, a CRISPR/CasX system, a CRISPR/CasY system, a CRISPR/Cascade system). Methods of genome editing to modify, delete, or insert nucleic acid sequences into genomic DNA are known in the art.

### Expression Constructs

Polynucleotides as described herein can be provided in an expression construct. Expression constructs generally include regulatory elements that are functional in the intended host cell in which the expression construct is to be expressed. Thus, a person of ordinary skill in the art can select regulatory elements for use in bacterial host cells, yeast host cells, plant host cells, insect host cells, mammalian host cells, and human host cells. Regulatory elements include promoters, transcription termination sequences, translation termination sequences, enhancers, and polyadenylation elements. As used herein, the term “expression construct” refers to a combination of nucleic acid sequences that provides for transcription of an operably linked nucleic acid sequence. As used herein, “operably linked” means two DNA molecules linked in manner so that one may affect the function of the other. Operably-linked DNA molecules may be part of a single contiguous molecule and may or may not be adjacent. For example, a promoter is operably linked with a polypeptide-encoding DNA molecule in a DNA construct where the two DNA molecules are so arranged that the promoter may affect the expression of the DNA molecule.

As used herein, the term “heterologous” refers to the relationship between two or more items derived from different sources and thus not normally associated in nature. For example, a protein-coding recombinant DNA molecule is heterologous with respect to an operably linked promoter if such a combination is not normally found in nature. In addition, a particular recombinant DNA molecule may be heterologous with respect to a cell, seed, or organism into which it is inserted when it would not naturally occur in that particular cell, seed, or organism.

An expression construct can comprise a promoter sequence operably linked to a polynucleotide sequence encoding a CYP81E polypeptide as described herein. Promoters can be incorporated into a polynucleotide using standard techniques known in the art. Multiple copies of promoters or multiple promoters can be used in an expression construct as described herein. In some embodiments, a promoter can be positioned about the same distance from the transcription start site in the expression construct as it is from the transcription start site in its natural genetic environment. Some variation in this distance is permitted without substantial decrease in promoter activity. A transcription start site is typically included in the expression construct.

Embodiments relate to a recombinant DNA molecule encoding a CYP81E polypeptide, wherein the recombinant DNA molecule is further defined as operably linked to a heterologous regulatory element. In specific embodiments, the heterologous regulatory element is a promoter functional in a plant cell. In further embodiments, the promoter is an inducible promoter.

If the expression construct is to be provided in or introduced into a plant cell, then plant viral promoters, such as, for example, a cauliflower mosaic virus (CaMV) 35S (including the enhanced CaMV 35S promoter (see, for example U.S. Pat. No. 5,106,739)) or a CaMV 19S promoter or a cassava vein mosaic can be used. Other promoters that can be used for expression constructs in plants include, for example, zein promoters including maize zein promoters, prolifera promoter, Ap3 promoter, heat shock promoters, T-DNA 1′- or 2′-promoter of A. tumefaciens, polygalacturonase promoter, chalcone synthase A (CHS-A) promoter from petunia, tobacco PR-la promoter, ubiquitin promoter, actin promoter, alcA gene promoter, pin2 promoter (Xu et al., 1993), maize Wip1 promoter, maize trpA gene promoter (U.S. Pat. No. 5,625,136), maize CDPK gene promoter, and RUBISCO SSU promoter (U.S. Pat. No. 5,034,322) can also be used. Constitutive promoters (such as the CaMV, ubiquitin, actin, or NOS promoter), developmentally-regulated promoters, and inducible promoters (such as those promoters than can be induced by heat, light, hormones, or chemicals) are also contemplated for use with polynucleotide expression constructs described herein.

Expression constructs may optionally contain a transcription termination sequence, a translation termination sequence, a sequence encoding a signal peptide, and/or enhancer elements. Transcription termination regions can typically be obtained from the 3′ untranslated region of a eukaryotic or viral gene sequence. Transcription termination sequences can be positioned downstream of a coding sequence to provide for efficient termination. A signal peptide sequence is a short amino acid sequence typically present at the amino terminus of a protein that is responsible for the relocation of an operably linked mature polypeptide to a wide range of post-translational cellular destinations, ranging from a specific organelle compartment to sites of protein action and the extracellular environment. Targeting gene products to an intended cellular and/or extracellular destination through the use of an operably linked signal peptide sequence is contemplated for use with the polypeptides described herein. Classical enhancers are cis-acting elements that increase gene transcription and can also be included in the expression construct.

Classical enhancer elements are known in the art, and include, but are not limited to, the CaMV 35S enhancer element, cytomegalovirus (CMV) early promoter enhancer element, and the SV40 enhancer element. Intron-mediated enhancer elements that enhance gene expression are also known in the art. These elements must be present within the transcribed region and are orientation dependent. Examples include the maize shrunken-1 enhancer element (Clancy and Hannah, 2002).

Optionally the gene encoding the CPY81E polypeptide is codon optimized to remove features inimical to expression and codon usage is optimized for expression in the particular crop (see, for example, U.S. Pat. No. 6,051,760; EP 0359472; EP 80385962; EP 0431829; and Perlak et al. (1991) PNAS USA 88:3324-3328; all of which are herein incorporated by reference).

In certain embodiments, the nucleic acid molecules include at least one nucleotide substitution, insertion, or deletion so that they do not recite a naturally occurring nucleic acid sequence.

### CYP81E Polypeptides

The terms “polypeptide” and “protein” are generally used interchangeably and refer to a single polypeptide chain which may or may not be modified by addition of non-amino acid groups. It would be understood that such polypeptide chains may associate with other polypeptides or proteins or other molecules such as co-factors. The terms “proteins” and “polypeptides” as used herein also include variants, mutants, modifications, analogous and/or derivatives of the polypeptides of the disclosure as described herein.

With regard to a defined polypeptide, it will be appreciated that % identity figures higher than those provided above will encompass preferred embodiments. Thus, where applicable, in light of the minimum % identity figures, it is preferred that the CPY81E polypeptide comprises an amino acid sequence which is at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to SEQ ID NO: 2.

By “variant” polypeptide is intended a polypeptide derived from the protein of SEQ ID NO: 2, by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

“Derivatives” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. Thus, functional variants and fragments of the CYP81E polypeptides, and nucleic acid molecules encoding them, also are within the scope of the present disclosure, and unless specifically described otherwise, irrespective of the origin of said polypeptide and irrespective of whether it occurs naturally

In addition, one of ordinary skill in the art will further appreciate that changes can be introduced by mutation into the nucleotide sequences thereby leading to changes in the amino acid sequence of the encoded proteins without altering the biological activity of the proteins. Thus, for example, an isolated polynucleotide molecule encoding a CYP81E polypeptide having an amino acid sequence that differs from that of SEQ ID NO: 2 can be created by introducing one or more nucleotide substitutions, additions, or deletions into the corresponding nucleotide sequence, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present disclosure. For example, preferably, conservative amino acid substitutions may be made at one or more predicted preferably nonessential amino acid residues. A “nonessential” amino acid residue is a residue that can be altered from the wild-type sequence of a protein without altering the biological activity, whereas an “essential” amino acid residue is required for biological activity.

A deletion refers to removal of one or more amino acids from a protein. An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag·100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues but may be clustered depending upon functional constraints placed upon the polypeptide and may range from 1 to 10 amino acids; insertions will usually be of the order of about 1 to 10 amino acid residues. A conservative amino acid substitution is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif. Conservative substitution tables are well known in the art (see for example Creighton (1984) Proteins. W.H. Freeman and Company (Eds).

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

In certain embodiments, the polypeptides include at least one amino acid substitution, insertion, or deletion so that they do not recite a naturally occurring amino acid sequence.

In certain embodiments, the CYP81E polypeptide comprises at least one of the following: an alanine residue at a position corresponding to position 9 of SEQ ID NO: 2; a serine residue at a position corresponding to position 12 of SEQ ID NO: 2; a histidine residue at a position corresponding to position 22 of SEQ ID NO: 2; a valine residue at a position corresponding to position 103 of SEQ ID NO: 2; a glycine residue at a position corresponding to position 157 of SEQ ID NO: 2; a serine residue at a position corresponding to position 258 of SEQ ID NO: 2; a threonine residue at a position corresponding to position 276 of SEQ ID NO: 2; a methionine residue at a position corresponding to position 379 of SEQ ID NO: 2; an alanine residue at a position corresponding to position 449 of SEQ ID NO: 2; a serine residue at a position corresponding to position 450 of SEQ ID NO: 2; an alanine residue at a position corresponding to position 463 of SEQ ID NO: 2; a valine residue at a position corresponding to position 489 of SEQ ID NO: 2; a leucine residue at a position corresponding to position 491 of SEQ ID NO: 2. The position of an amino acid residue in a given amino acid sequence is typically numbered herein using the numbering of the position of the corresponding amino acid residue of the Amaranthus tuberculatus CYP81E amino acid sequence shown in SEQ ID NO:2.

“Orthologs” and “paralogs” encompass evolutionary concepts used to describe the ancestral relationships of genes. Paralogs are genes within the same species that have originated through duplication of an ancestral gene; orthologs are genes from different organisms that have originated through speciation, and are also derived from a common ancestral gene.

Orthologs and paralogs of SEQ ID NO: 2 encompassed by the present disclosure include, but are not limited to, polypeptides comprising SEQ ID NO: 33, 34, 35, 36, 37, 38, 39, 40, 41, 42, 43, or 44.

TABLE 1

| Protein | Species | Amino acid sequence |
|---|---|---|
| Spov3_chr3.03506 | Spinacia oleracea | SEQ ID NO: 33 |
| EL10Ac3g07035.1 | Beta vulgaris | SEQ ID NO: 34 |
| AUR62024416-RA | Chenopodium quinoa | SEQ ID NO: 35 |
| Ciclev10025420m.g | Citrus Clementina | SEQ ID NO: 36 |
| Glyma.16G149300 | Glycine max | SEQ ID NO: 37 |
| Solyc04g078360.1 | Solanum lycopersicum | SEQ ID NO: 38 |
| HanXRQChr02g0040021 | Helianthus annuus | SEQ ID NO: 39 |
| Soltu.DM.04G033120 | Solanum tuberosum | SEQ ID NO: 40 |
| Prupe.6G227100 | Prunus persica | SEQ ID NO: 41 |
| Gohir.D12G056000 | Gossypium hirsutum | SEQ ID NO: 42 |
| Manes.13G116700 | Manihot esculenta | SEQ ID NO: 43 |
| Sialb.0008s1384 | Sinapis alba | SEQ ID NO: 44 |

### Transformation Methods

Several embodiments relate to plant cells, plant tissues, plants, and seeds that comprise a recombinant DNA as described herein. In some embodiments, cells, tissues, plants, and seeds comprising the recombinant DNA molecules exhibit tolerance to auxin herbicides.

Suitable methods for transformation of host plant cells include virtually any method by which DNA or RNA can be introduced into a cell (for example, where a recombinant DNA construct is stably integrated into a plant chromosome or where a recombinant DNA construct or an RNA is transiently provided to a plant cell) and are well known in the art. Two effective methods for cell transformation are Agrobacterium-mediated transformation and microprojectile bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616, which is incorporated herein by reference in its entirety. Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores and pollen. Callus can be initiated from tissue sources including, but not limited to, immature or mature embryos, hypocotyls, seedling apical meristems, microspores and the like. Cells containing a transgenic nucleus are grown into transgenic plants.

In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes. Preferred marker genes provide selective markers which confer resistance to a selective agent, such as an antibiotic or an herbicide. Any of the herbicides to which plants of this disclosure can be resistant is an agent for selective markers. Potentially transformed cells are exposed to the selective agent. In the population of surviving cells are those cells where, generally, the resistance-conferring gene is integrated and expressed at sufficient levels to permit cell survival. Cells can be tested further to confirm stable integration of the exogenous DNA. Commonly used selective marker genes include those conferring resistance to antibiotics such as kanamycin and paromomycin (nptII), hygromycin B (aph IV), spectinomycin (aadA) and gentamycin (aac3 and aacC4) or resistance to herbicides such as glufosinate (bar or pat), dicamba (DMO) and glyphosate (aroA or EPSPS). Examples of such selectable markers are illustrated in U.S. Pat. Nos. 5,550,318; 5,633,435; 5,780,708 and 6,118,047. Markers which provide an ability to visually screen transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Plants with Herbicide Tolerance

Several embodiments relate to plant cells, plant tissues, plants, and seeds that comprise a polynucleotide encoding a CYP81E polypeptide, wherein expression of the polynucleotide confers tolerance to an herbicide. Plants may be monocots or dicots, and may include, for example, rice, wheat, barley, oats, rye, sorghum, maize, grape, tomato, potato, lettuce, broccoli, cucumber, peanut, melon, pepper, carrot, squash, onion, soybean, alfalfa, sunflower, cotton, canola, and sugar beet plants.

Plants that are particularly useful in the methods of the present disclosure include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. In certain embodiments, the plant is a crop plant. Examples of crop plants include inter alia soybean, sunflower, canola, alfalfa, rapeseed, cotton, tomato, potato, or tobacco.

Certain embodiments encompass a progeny or a descendant of an herbicide-tolerant plant as well as seeds derived from the herbicide-tolerant plants and cells derived from the herbicide-tolerant plants as described herein.

In some embodiments, the present disclosure provides a progeny or descendant plant derived from a plant comprising in at least some of its cells a polynucleotide operably linked to a promoter functional in a plant cell, the promoter capable of expressing a CPY81E polypeptide encoded by the polynucleotide, wherein the progeny or descendant plant comprises in at least some of its cells the recombinant polynucleotide operably linked to the promoter, the expression of the CYP81E polypeptide conferring to the progeny or descendant plant tolerance to the herbicide.

In one embodiment, seeds of the present disclosure preferably comprise the herbicide-tolerance characteristics of the herbicide-tolerant plant. In other embodiments, a seed is capable of germination into a plant comprising in at least some of its cells a polynucleotide operably linked to a promoter functional in a plant cell, the promoter capable of expressing a CYP81E polypeptide encoded by the polynucleotide, the expression of the CPY81E polypeptide conferring to the progeny or descendant plant tolerance to the herbicides.

In some embodiments, plant cells of the present disclosure are capable of regenerating a plant or plant part. In other embodiments, plant cells are not capable of regenerating a plant or plant part. Examples of cells not capable of regenerating a plant include, but are not limited to, endosperm, seed coat (testa and pericarp), and root cap.

In another embodiment, the disclosure refers to a plant cell transformed by a nucleic acid encoding a CPY81E polypeptide as described herein, wherein expression of the nucleic acid in the plant cell results in increased resistance or tolerance to an herbicide as compared to a wild type variety of the plant cell.

Several embodiments provide a plant product prepared from the herbicide-tolerant plants. In some embodiments, examples of plant products include, without limitation, grain, oil, and meal. In one embodiment, a plant product is plant grain (e.g., grain suitable for use as feed or for processing), plant oil (e.g., oil suitable for use as food or biodiesel), or plant meal (e.g., meal suitable for use as feed). A preferred plant product is fodder, seed meal, oil, or seed-treatment-coated seeds. Preferably, the meal and/or oil comprise the CYP81E nucleic acid or CYP81E protein.

In certain embodiments, a plant product prepared from a plant or plant part is provided, wherein the plant or plant part comprises in at least some of its cells a polynucleotide operably linked to a promoter functional in plant cells, the promoter capable of expressing a CYP81E polypeptide encoded by the polynucleotide, the expression of the CYP81E polypeptide conferring to the plant or plant part tolerance to the herbicide.

The product may be produced at the site where the plant has been grown, the plants and/or parts thereof may be removed from the site where the plants have been grown to produce the product. Typically, the plant is grown, the desired harvestable parts are removed from the plant, if feasible in repeated cycles, and the product made from the harvestable parts of the plant. The step of growing the plant may be performed only once each time the method is performed, while allowing repeated times the steps of product production e.g. by repeated removal of harvestable parts of the plants of the disclosure and if necessary further processing of these parts to arrive at the product. It is also possible that the step of growing the plants is repeated and plants or harvestable parts are stored until the production of the product is then performed once for the accumulated plants or plant parts. Also, the steps of growing the plants and producing the product may be performed with an overlap in time, even simultaneously to a large extend or sequentially. Generally, the plants are grown for some time before the product is produced.

Auxin Herbicides

Synthetic auxin herbicides are also called auxinic, growth regulator herbicides, or Group O or Group 4 herbicides, based on their mode of action. The mode of action of the synthetic auxin herbicides is that they appear to affect cell wall plasticity and nucleic acid metabolism, which can lead to uncontrolled cell division and growth. The group of synthetic auxin herbicides includes four chemical families: phenoxy, carboxylic acid (or pyridine), benzoic acid, and the newest family quinoline carboxylic acids.

The phenoxy herbicides are most common and have been used as herbicides since the 1940s when (2,4-dichlorophenoxy) acetic acid (2,4-D) was discovered. Other examples include 4-(2,4-dichlorophenoxy) butyric acid (2,4-DB), 2-(2,4-dichlorophenoxy) propanoic acid (2, 4-DP), (2,4,5-trichlorophenoxy)acetic acid (2,4,5-T), 2-(2,4,5-Trichlorophenoxy) Propionic Acid (2,4,5-TP), 2-(2,4-dichloro-3-methylphenoxy)-N-phenylpropanamide (clomeprop), (4-chloro-2-methylphenoxy) acetic acid (MCPA), 4-(4-chloro-o-tolyloxy) butyric acid (MCPB), and 2-(4-chloro-2-methylphenoxy) propanoic acid (MCPP).

The next largest chemical family is the carboxylic acid herbicides, also called pyridine herbicides. Examples include 3,6-dichloro-2-pyridinecarboxylic acid (Clopyralid), 4-amino-3,5,6-trichloro-2-pyridinecarboxylic acid (picloram), (2,4,5-trichlorophenoxy) acetic acid (triclopyr), and 4-amino-3,5-dichloro-6-fluoro-2-pyridyloxyacetic acid (fluroxypyr). The third chemical family is the benzoic acids, examples of which include 3,6-dichloro-o-anisic acid (dicamba) and 3-amino-2,5-dichlorobenzoic acid (choramben). The fourth and newest chemical family of the auxinic herbicides is the quinaline carboxylic acid family, which includes 7-chloro-3-methyl-8-quinolinecarboxylic acid (quinmerac) and 3,7-dichloro-8-quinolinecarboxylic acid (quinclorac). This latter is unique in that it also will control some grass weeds, unlike the other auxin-like herbicides which essentially control only broadleaf or dicotyledonous plants.

Synthetic auxin herbicides may be applied to a plant growth area comprising the plants and seeds provided by the compositions and methods described herein as a method for controlling weeds. Plants and seeds provided by the compositions and methods described herein comprise a synthetic auxin herbicide tolerance trait and as such are tolerant to the application of one or more auxin herbicides. The herbicide application may be the recommended commercial rate (1×) or any fraction or multiple thereof, such as twice the recommended commercial rate (2×). Auxin herbicide rates may be expressed as acid equivalent per pound per acre (lb ae/acre) or acid equivalent per gram per hectare (g ae/ha) or as pounds active ingredient per acre (lb ai/acre) or grams active ingredient per hectare (g ai/ha), depending on the herbicide and the formulation. The plant growth area may or may not comprise weed plants at the time of herbicide application.

Herbicide applications may be sequentially or tank mixed with one, two, or a combination of several auxin herbicides or any other compatible herbicide. Multiple applications of one herbicide or of two or more herbicides, in combination or alone, may be used over a growing season to areas comprising plants expressing CYP81E protein as described herein for the control of a broad spectrum of dicot weeds, monocot weeds, or both, for example, two applications (such as a pre-planting application and a post-emergence application or a pre-emergence application and a post-emergence application) or three applications (such as a pre-planting application, a pre-emergence application, and a post-emergence application or a pre-emergence application and two post-emergence applications).

### Herbicide Resistant Weed Control

Several embodiments provide compositions and methods for controlling the growth of an herbicide resistant weed at a plant cultivation site by contacting the weed with a composition comprising a polynucleotide that reduces expression or activity of a CYP81E polypeptide.

Systemic regulation (e.g., systemic suppression or silencing) of a target CYP81E gene in a plant can be by topical application to the plant of a polynucleotide molecule with a segment in a nucleotide sequence essentially identical to, or essentially complementary to, a sequence of 18 or more contiguous nucleotides in either the target CYP81E gene or RNA transcribed from the target CYP81E gene, whereby the composition permeates the interior of the plant and induces systemic regulation of the target CYP81E gene by the action of single-stranded RNA that hybridizes to the transcribed RNA, e.g., messenger RNA.

The polynucleotides are designed to induce systemic regulation or suppression of an endogenous gene in a plant and are designed to have a sequence essentially identical or essentially complementary to the sequence (which can be coding sequence or non-coding sequence) of an endogenous CYP81E gene of a resistant plant or to the sequence of RNA transcribed from an endogenous CYP81E gene of a resistant plant. By “essentially identical” or “essentially complementary” is meant that the polynucleotides (or at least one strand of a double-stranded polynucleotide) are designed to hybridize under physiological conditions in cells of the plant to the endogenous gene or to RNA transcribed from the endogenous gene to effect regulation or suppression of the endogenous gene.

In certain embodiments, the compositions and methods can comprise permeability-enhancing agents and treatments to condition the surface of plant tissue, e.g., leaves, stems, roots, flowers, or fruits, to permeation by the polynucleotides into plant cells. The transfer of polynucleotides into plant cells can be facilitated by the prior or contemporaneous application of a polynucleotide to the plant tissue. In some embodiments the permeability-enhancing agent is applied subsequent to the application of the polynucleotide composition. The permeability-enhancing agent enables a pathway for polynucleotides through cuticle wax barriers, stomata and/or cell wall or membrane barriers and into plant cells. Suitable agents to facilitate transfer of the composition into a plant cell include agents that increase permeability of the exterior of the plant or that increase permeability of plant cells to oligonucleotides or polynucleotides. Such agents to facilitate transfer of the composition into a plant cell include a chemical agent, or a physical agent, or combinations thereof.

Chemical agents for conditioning include (a) surfactants, (b) an organic solvent or an aqueous solution or aqueous mixtures of organic solvents, (c) oxidizing agents, (e) acids, (f) bases, (g) oils, (h) enzymes, or combinations thereof. Embodiments of the method can optionally include an incubation step, a neutralization step (e.g., to neutralize an acid, base, or oxidizing agent, or to inactivate an enzyme), a rinsing step, or combinations thereof. Such agents for conditioning of a plant to permeation by polynucleotides are applied to the plant by any convenient method, e.g., spraying or coating with a powder, emulsion, suspension, or solution; similarly, the polynucleotide molecules are applied to the plant by any convenient method, e.g., spraying or wiping a solution, emulsion, or suspension.

### Detection Tools

Several embodiments provide a method for identifying an herbicide-resistant plant, or cells or tissues thereof. In some embodiments, the method includes using primers or probes which specifically recognize a portion of the sequence of the gene. In an embodiment, the method is based on identifying the expression level of a CPY81E gene in the plant. In some embodiments, a PCR-based technique is used to quantify the expression of a CPY81E gene that is differentially expressed in resistant plants compared to sensitive plants prior to treatment. In other words, basal expression levels are heightened in resistant plants compared to sensitive plants prior to herbicide treatment.

In some embodiments, the identification is performed using polymerase chain reaction. The method may also include providing a detectable marker specific to the CYP81E gene. In embodiments, the detection is performed using an Enzyme-Linked Immunosorbent Assay (ELISA), a quantitative real-time polymerase chain reaction (qPCR), or an RNA-hybridization technique.

In one embodiment, the method is based on the presence of SNPs between S and R plants. This can be based on fluorescent detection of SNP-specific hybridization probes on PCR products such as Taqman or Molecular Beacons. Other strategies such as Sequenom homogeneous Mass Extend (hME) and iPLEX genotyping systems involve MALDI-TOF mass spectrophotometry of SNP-specific PCR primer extension products.

Other methods employ include use of KASP™, that is, Kompetitive Allele Specific PCR. It is based on competitive allele-specific PCR and allows scoring of single nucleotide polymorphisms (SNPs), as well as deletions and insertions at specific loci. Two allele specific forward primers are used having the target SNP at the 3′ end and a common reverse primer is used for both. The primers have a unique “tail” sequence (reporter nucleotide sequence) compatible with a different fluorescent reporter (reporter molecule). The primers are contacted with the sample along with a mix which includes a universal Fluorescence Resonant Energy Transfer (FRET) cassette and Taq polymerase. During rounds of PCR cycling, the tail sequences allow the FRET cassette to bind to the DNA and emit fluorescence. See, e.g. Yan et al. “Introduction of high throughput and cost effective SNP genotyping platforms in soybean” Plant Genetics, Genomic and Biotechnology 2(1): 90-94 (2014); Semagn et al. “Single nucleotide polymorphism genotyping using Kompetitive Allele Specific PCR (KASP): overview of the technology and its application in crop improvement” Molecular Breeding 33(1): 1-14 (2013). In the present process, emission of one fluorescent signal (reporter molecule) or the other indicates the plant is one of the two species, where presence of both signals indicates a hybrid. Examples here show use of 6-carboxyflurescein (FAM); and 6-carboxy-2′,4,4′,5′,7,7′-hexachlorofluorescein (HEX) fluorophores, however any convenient means of producing a measurable signal may be used. Examples without intending to be limiting include tetrachlorofluorescein (TET); cyan florescent protein, yellow fluorescent protein, luciferase, SyBR Green I; ViC; CAL Fluor Gold 540, ROX Texas Red; CAL Fluor Red 610; CY5; Quasar 670; Quasar 705; and Fret.

In sum, a first primer is produced recognizing a first target nucleotide sequence in the genome of a first species, a second primer is produced recognizing a second target nucleotide sequence of a second species and the third common reverse primer universal to all genotypes allows for amplification. A “tail” reporter sequence is provided with the primer. The expression cassette comprises sequences complementary to the reporter sequence. With rounds of PCR, the cassette is no longer quenched and a measurable signal is produced.

Two sets of KASP primers designed on the location of CPY81E are set forth in SEQ ID NOs: 27-29 and 30-31. Primers for R alleles were tagged with HEX fluorophore and S with FAM.

TABLE 2

| | |
|---|---|
| 1 Forward_HEX (R) | GAAGGTCGGAGTCAACGGATTCCAATCTCTAG |
| | CCCAACGTTACGGT (SEQ ID NO: 27) |
| 1 Forward_FAM (S) | GAAGGTGACCAAGTTCATGCTCCAATCTCTAG |
| | CCCAACGTTACGGC (SEQ ID NO: 28) |
| 1 Reverse (5′-3′) | CAACGGGCCTTGGTAGTTTC |
| | (SEQ ID NO: 29) |
| 2 Forward_HEX (R) | GAAGGTCGGAGTCAACGGATTTTTGCAAACAG |
| | ACCAAAATTCATAGTAGGC |
| | (SEQ ID NO: 30) |
| 2 Forward _FAM | GAAGGTGACCAAGTTCATGCTTTTGCAAACAG |
| (S) | ACCAAAATTCATAATAGGA |
| | (SEQ ID NO: 31) |
| 2 Reverse (5′-3′) | CTTATGGGGACTACTGGCGG |
| | (SEQ ID NO: 32) |

Several embodiments provide kits for identifying herbicide-resistant plants, the kits comprising at least two primers or probes that specifically recognize the CYP81E gene. For example, primers have been developed to amplify and/or quantify the expression of the CYP81E gene associated with SEQ ID NO: 1. By evaluating the expression level of the gene, one skilled in the art is able to determine whether a plant sample comes from an herbicide-resistant plant. In certain embodiments, the primers comprise SEQ ID NOs: 5 and 6. Kits for detecting the presence of a SNP between S and R plants are also provided. In certain embodiments, the primers comprise SEQ ID NOs: 27-29 or 30-32. In an embodiment, the kit includes more than one primer pair. The kit may also include one or more positive or negative controls.

In some embodiments, the kits include a specific probe having a sequence which corresponds to or is complementary to a sequence having between 80% and 100% sequence identity with a specific region of the CYP81E gene. In some embodiments, the kit includes a specific probe which corresponds to or is complementary to a sequence having between 90% and 100% sequence identity with a specific region of the CPY81E gene.

The methods, kits, and primers can be used for different purposes including, but not limited to the following: identifying the presence or absence of herbicide resistance in plants, plant material such as seeds or cuttings; determining the presence of herbicide-resistant weeds in crop fields; and tailoring an herbicide regime to effectively and economically manage weeds affecting agricultural crops.

### Use in Breeding Methods

The plants of the disclosure may be used in a plant breeding program. The goal of plant breeding is to combine, in a single variety or hybrid, various desirable traits. For field crops, these traits may include, for example, resistance to diseases and insects, tolerance to heat and drought, tolerance to chilling or freezing, reduced time to crop maturity, greater yield and better agronomic quality. With mechanical harvesting of many crops, uniformity of plant characteristics such as germination and stand establishment, growth rate, maturity and plant and ear height is desirable. Traditional plant breeding is an important tool in developing new and improved commercial crops. This disclosure encompasses methods for producing a plant by crossing a first parent plant with a second parent plant wherein one or both of the parent plants is a plant displaying a phenotype as described herein.

Plant breeding techniques known in the art and used in a plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids and transformation. Often combinations of these techniques are used.

The development of hybrids in a plant breeding program requires, in general, the development of homozygous inbred lines, the crossing of these lines and the evaluation of the crosses. There are many analytical methods available to evaluate the result of a cross. The oldest and most traditional method of analysis is the observation of phenotypic traits. Alternatively, the genotype of a plant can be examined.

A genetic trait which has been engineered into a particular plant using transformation techniques can be moved into another line using traditional breeding techniques that are well known in the plant breeding arts. For example, a backcrossing approach is commonly used to move a transgene from a transformed plant to an elite inbred line and the resulting progeny would then comprise the transgene(s). Also, if an inbred line was used for the transformation, then the transgenic plants could be crossed to a different inbred in order to produce a transgenic hybrid plant. As used herein, “crossing” can refer to a simple X by Y cross or the process of backcrossing, depending on the context.

The development of a hybrid in a plant breeding program involves three steps: (1) the selection of plants from various germplasm pools for initial breeding crosses; (2) the selfing of the selected plants from the breeding crosses for several generations to produce a series of inbred lines, which, while different from each other, breed true and are highly homozygous and (3) crossing the selected inbred lines with different inbred lines to produce the hybrids. During the inbreeding process, the vigor of the lines decreases. Vigor is restored when two different inbred lines are crossed to produce the hybrid. An important consequence of the homozygosity and homogeneity of the inbred lines is that the hybrid created by crossing a defined pair of inbreds will always be the same. Once the inbreds that give a superior hybrid have been identified, the hybrid seed can be reproduced indefinitely as long as the homogeneity of the inbred parents is maintained.

Plants of the present disclosure may be used to produce, e.g., a single cross hybrid, a three-way hybrid or a double cross hybrid. A single cross hybrid is produced when two inbred lines are crossed to produce the F1 progeny. A double cross hybrid is produced from four inbred lines crossed in pairs (A×B and C×D) and then the two F1 hybrids are crossed again (A×B) times (C×D). A three-way cross hybrid is produced from three inbred lines where two of the inbred lines are crossed (A×B) and then the resulting F1 hybrid is crossed with the third inbred (A×B)×C. Much of the hybrid vigor and uniformity exhibited by F1 hybrids is lost in the next generation (F2). Consequently, seed produced by hybrids is consumed rather than planted.

### Embodiments

The following numbered embodiments also form part of the present disclosure:

1. A modified plant, or a progeny, plant part, or plant cell thereof, having tolerance to an herbicide, the modified plant comprising increased expression of a polynucleotide encoding a cytochrome P450 81E (CYP81E) polypeptide relative to an unmodified plant.

2. The modified plant of embodiment 1, wherein the modified plant comprises a heterologous polynucleotide encoding the CYP81E polypeptide.

3. The modified plant of embodiment 1 or embodiment 2, wherein the CYP81E polypeptide has at least 80%, at least 90%, at least 95%, at least 98%, or at least 99% sequence identity to SEQ ID NO: 2.

4. The modified plant of any one of embodiments 1-3, wherein the polynucleotide encoding the CYP81E polypeptide has at least 80%, at least 90%, at least 95%, at least 98%, or at least 99% sequence identity to SEQ ID NO: 1.

5. The modified plant of any one of embodiments 1-4, wherein the CYP81E polypeptide has at least 80%, at least 90%, at least 95%, at least 98%, or at least 99% sequence identity to any of SEQ ID NOs: 33-44.

6. The modified plant of any one of embodiments 1-5, wherein the polynucleotide is operably linked to a promoter functional in a plant cell.

7. The modified plant of any one of embodiments 1-6, wherein the herbicide is an auxin herbicide.

8. The modified plant of any one of embodiments 1-7, wherein the auxin herbicide is 2,4-D.

9. The modified plant of any one of embodiments 1-8, wherein the plant is dicotyledonous.

10. The modified plant of any one of embodiments 1-9, wherein the plant is a crop plant.

11. The modified plant of any one of embodiments 1-10, wherein the plant is a soybean, cotton, canola, tobacco, tomato, potato, alfalfa, sugar beet, or sunflower plant.

12. The modified plant of any one of embodiments 1-11, wherein the modified plant further comprises a second herbicide-tolerant trait.

13. A nucleic acid molecule comprising a nucleotide sequence selected from: (a) a nucleotide sequence encoding a CYP81E polypeptide, wherein the nucleotide sequence has at least 80%, at least 90%, at least 95%, at least 98%, or at least 99% sequence identity to SEQ ID NO: 1; or (b) a nucleotide sequence encoding a CYP81E polypeptide, wherein the CYP81E polypeptide has at least 80%, at least 90%, at least 95%, at least 98%, or at least 99% sequence identity to SEQ ID NO: 2.

14. The nucleic acid molecule of embodiment 13, wherein the nucleic acid molecule is an isolated, synthetic, or recombinant nucleic acid molecule.

15. An expression cassette comprising the nucleic acid molecule of embodiment 13 or embodiment 14 operably linked to a heterologous promoter functional in a plant cell.

16. A vector comprising the nucleic acid molecule of embodiment 13 or embodiment 14; or the expression cassette of embodiment 15.

17. A CYP81E polypeptide comprising an amino acid sequence having at least 80%, at least 90%, at least 95%, at least 98%, or at least 99% sequence identity to SEQ ID NO: 2.

18. A plant, plant part, or plant cell comprising the nucleic acid molecule of embodiment 13 or embodiment 14; the expression cassette of embodiment 15; the vector of embodiment 16; or the polypeptide of embodiment 17.

19. A biological sample comprising the nucleic acid molecule of embodiment 13 or embodiment 14; the expression cassette of embodiment 15; the vector of embodiment 16; or the polypeptide of embodiment 17.

20. A method for producing a plant with herbicide tolerance, the method comprising: increasing expression of a polynucleotide encoding a CYP81E polypeptide in the plant, wherein the herbicide tolerance of the plant is increased when compared to a plant that lacks the increased expression.

21. The method of embodiment 20 comprising introducing to a plant cell a polynucleotide encoding the CYP81E polypeptide, wherein the polynucleotide is operably linked to a heterologous promoter functional in a plant cell; and regenerating a plant from the plant cell.

22. The method of embodiment 20 or embodiment 21, wherein the CYP81E polypeptide has at least 80%, at least 90%, at least 95%, at least 98%, or at least 99% sequence identity to SEQ ID NO: 2.

23. The method of any one of embodiments 20-22, wherein the polynucleotide encoding the CYP81E polypeptide has at least 80%, at least 90%, at least 95%, at least 98%, or at least 99% sequence identity to SEQ ID NO: 1.

24. The method of any one of embodiments 20-23, wherein the CYP81E polypeptide has at least 80%, at least 90%, at least 95%, at least 98%, or at least 99% sequence identity to any of SEQ ID NOs: 33-44.

25. The method of any one of embodiments 20-24, wherein the herbicide is an auxin herbicide.

26. The method of any one of embodiments 20-25, wherein the auxin herbicide is 2,4-D.

27. The method of any one of embodiments 20-26, wherein the plant is dicotyledonous.

28. The method of any one of embodiments 20-27, wherein the plant is a crop plant.

29. The method of any one of embodiments 20-28, wherein the plant a soybean, cotton, canola, tobacco, tomato, potato, alfalfa, sugar beet, or sunflower plant.

30. A method for controlling undesired vegetation at a plant cultivation site, the method comprising: providing at the site a plant that comprises a polynucleotide encoding a CYP81E polypeptide, wherein expression of the polynucleotide confers to the plant tolerance to an herbicide; and applying to the site an effective amount of the herbicide.

31. The method of embodiment 30, wherein the CYP81E polypeptide has at least 80%, at least 90%, at least 95%, at least 98%, or at least 99% sequence identity to SEQ ID NO: 2.

32. The method of embodiment 30 or embodiment 31, wherein the polynucleotide encoding the CYP81E polypeptide has at least 80%, at least 90%, at least 95%, at least 98%, or at least 99% sequence identity to SEQ ID NO: 1.

33. The method of any one of embodiments 30-32, wherein the polynucleotide is operably linked to a heterologous promoter functional in a plant cell.

34. The method of any one of embodiments 30-33, wherein the herbicide is an auxin herbicide.

35. The method of any one of embodiments 30-34, wherein the auxin herbicide is 2,4-D.

36. The method of any one of embodiments 30-35, wherein the plant is dicotyledonous.

37. The method of any one of embodiments 30-36, wherein the plant a soybean, cotton, canola, tobacco, tomato, potato, alfalfa, sugar beet, or sunflower plant.

38. A method for controlling the growth of an herbicide resistant weed at a plant cultivation site, the method comprising: contacting the weed with a composition comprising a polynucleotide that reduces expression or activity of a CYP81E polypeptide; and applying to the site an effective amount of the herbicide.

39. The method of embodiment 38, wherein the polynucleotide is a double-stranded RNA, a single-stranded RNA, or a double-stranded DNA/RNA hybrid polynucleotide.

40. The method of embodiment 38 or embodiment 39, wherein the polynucleotide comprises a sequence essentially identical or essentially complementary to at least 18 or more contiguous nucleotides of SEQ ID NO: 1.

41. The method of any one of embodiments 38-40, wherein the polynucleotide has a length of 26-60 nucleotides.

42. The method of any one of embodiments 38-41, wherein the CYP81E polypeptide has at least 80%, at least 90%, at least 95%, at least 98%, or at least 99% sequence identity to SEQ ID NO: 2.

43. The method of any one of embodiments 38-42, wherein the herbicide is an auxin herbicide.

44. The method of any one of embodiments 38-43, wherein the auxin herbicide is 2,4-D.

45. The method of any one of embodiments 38-44, wherein the weed is Amaranthus tuberculatus.

46. The method of any one of embodiments 38-45, wherein the composition comprises an agent that enables the polynucleotide to permeate from the surface of the weed into cells of the weed.

47. A product prepared from the plant, plant part, or plant cell of any one of embodiments 1-12, wherein the product comprises the polynucleotide encoding the CYP81E polypeptide.

48. The product of embodiment 47, wherein the product is fodder, seed meal, oil, or seed-treatment-coated seed.

49. A method for producing a plant product, the method comprising processing the plant or plant part of any one of embodiments 1-12 to obtain the plant product, wherein the plant product comprises the polynucleotide encoding the CYP81E polypeptide.

50. The method of embodiment 49, wherein the plant product is fodder, seed meal, oil, or seed-treatment-coated seeds.

51. A method for identifying an herbicide-resistant plant, the method comprising: providing a biological sample from a plant suspected of having herbicide resistance; quantifying expression of a CYP81E gene in the biological sample, wherein the CYP81E gene is differentially expressed in an herbicide-resistant plant compared to an herbicide-sensitive plant of the same species; and determining that the plant is herbicide-resistant based on the quantification.

52. The method of embodiment 51, wherein the biological sample is from Amaranthus tuberculatus.

53. The method of embodiment 51 or embodiment 52, wherein the herbicide is an auxin herbicide.

54. The method of any one of embodiments 51-53, wherein the quantifying expression of the CYP81E gene comprises quantifying CYP81E mRNA.

55. The method of any one of embodiments 51-54, wherein the quantifying expression of the CYP81E gene comprises quantifying CYP81E polypeptide.

56. The method of any one of embodiments 51-55, wherein the CYP81E gene has at least four-fold differential expression in the herbicide-resistant plant compared to the herbicide-sensitive plant prior to application of the herbicide.

57. The method of any one of embodiments 51-56, wherein the CYP81E gene has at least 80%, at least 90%, at least 95%, at least 98%, or at least 99% sequence identity to SEQ ID NO: 1.

58. The method of any one of embodiments 51-57, wherein the quantifying expression comprises amplifying a nucleic acid using at least two primers.

59. The method of any one of embodiments 51-58, wherein the at least two primers comprise SEQ ID NO: 5 and SEQ ID NO: 6.

60. A kit for identifying an herbicide-resistant plant, the kit comprising at least two primers, wherein the at least two primers recognize a CYP81E gene that is differentially expressed in an herbicide-resistant plant compared to an herbicide-sensitive plant of the same species.

61. The kit of embodiment 60, wherein the wherein the CYP81E gene has at least 80%, at least 90%, at least 95%, at least 98%, or at least 99% sequence identity to SEQ ID NO: 1.

62. The kit of embodiment 60 or embodiment 61, further comprising at least one of a positive control and a negative control.

63. The kit of any one of embodiments 60-62, further comprising components of a qRT-PCR solution.

64. The kit of any one of embodiments 60-63, wherein the plant is Amaranthus tuberculatus and the herbicide is an auxin herbicide.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

### Example 1: Resistance Response

Two populations of A. tuberculatus showing resistance to HPPD inhibitors and 2,4-D were identified from both Illinois (referred to as “CHR”) (Evans et al. 2019) and Nebraska (referred to as “NEB”) (Bernards et al. 2012). Herbicide-resistant plants from each population were crossed with an herbicide-sensitive A. tuberculatus population (WUS; originally collected in Brown County, Ohio) and F₁ seeds were screened to confirm resistance to both HPPD inhibitors and 2,4-D. To screen these F₁ populations, plants were grown under previously described greenhouse conditions (Lillie et al., 2020) and sprayed with an initial discriminating dose of mesotrione (220 g ai ha⁻¹; Callisto) plus 1% v/v crop oil concentrate, followed by a late POST treatment of 2,4-D (560 g ae ha⁻¹; 2,4-D amine) plus 0.25% v/v nonionic surfactant. All herbicide applications were made using a moving-nozzle spray chamber as described previously (Lillie et al. 2020). Within each of the NEB- and CHR-derived F₁ lines, pairs of full-sibling F₁ survivors were crossed together to form several segregating pseudo-F₂ populations. Because A. tuberculatus is dioecious, an F₁ plant cannot be selfed to create a true F₂ population.

A single pseudo-F₂ (hereafter referred to as an F₂) population was selected each from NEB and CHR and several hundred seeds from each F₂ were germinated for 48 hours on wet filter paper in a growth chamber set to a 12-hr day/night cycle (35° C./15° C.). Germinated seedlings were transplanted into 50-cm³ pots filled with Weed Lite Mix (3:1:1:1 mixture of LC1 [Sun Gro Horticulture Canada]:Soil:Peat:Torpedo Sand) and grown in the greenhouse until plants reached a height of 4-6 cm. One hundred plants from each F₂ population were then transplanted into 3.8-L round pots filled with Weed Lite Mix and allowed to grow until plants reached 8-10 cm in height. Tissue was then collected from the smallest fully unfolded leaf, immediately placed into liquid nitrogen, and stored at −80° C. until RNA extraction. All tissue was collected within a two-hour period between 10 am and noon on the same day. Tissue was taken prior to herbicide application and herbicide-treated tissue was not included in this study. Without the use of an extensive (and expensive) time course RNAseq study, identifying potential resistance genes that are induced by herbicide application is extremely difficult due to the differential effects of herbicide treatment on stress and death pathways between resistant and sensitive plants (Giacomini et al., 2018).

All F₂ plants continued to grow for three more weeks until each plant had produced multiple side shoots, at which point the side shoots were clipped off, dipped in rooting hormone, and transplanted into 400-cm³ inserts in flats filled with damp soil. These flats were covered with a clear 15-cm plastic dome (to maintain high humidity) until the clones established a good root system (˜3-4 weeks). Four clones were produced from each plant and each clone was treated with either an HPPD inhibitor or 2,4-D at a high or low dose to phenotype each F₂ individual for multiple herbicide resistance. The low and high rates of HPPD inhibitor were 27 and 270 g tembotrione (Laudis), respectively. The low and high rates of 2,4-D were 560 and 2240 g ae ha⁻¹ (2,4-D amine), respectively. Clones were visually rated for herbicide damage 14 and 21 DAT, using a 1-10 scale (a score of 10 indicated no plant damage).

The cloning and spraying procedure was repeated on another 70 plants from each population to generate enough data for a Fisher's exact test to assess whether the two resistance traits segregated independently of one another. Using a cut-off of 3 on the visual rating scale to score plants as either sensitive or resistant, count data for each category was fed into R and analyzed using fisher.test (alternative=“two.sided”).

Based on the clonal visual ratings at both rates 21 DAT, F₂ plants were ranked in order of least to most resistant for both tembotrione and 2,4-D. Within each F₂ population, plants were then grouped into four categories: (1) RR, resistant to both 2,4-D and tembotrione; (2) RS, resistant to 2,4-D and sensitive to tembotrione; (3) SR, sensitive to 2,4-D and resistant to tembotrione; and (4) SS, sensitive to both 2,4-D and tembotrione. The four most resistant and sensitive in each category (sixteen plants total from each population and 32 plants overall) were selected for RNA extraction using a Trizol-based method (Simms et al. 1993) with a DNase I treatment following extraction. Samples were checked for quality and quantity, respectively, by running them on a Qubit analyzer and on a 1% agarose gel before sending them to the Roy J. Carver Biotechnology Center at the University of Illinois, Urbana-Champaign for Illumina library construction and sequencing.

The RNAseq libraries were prepared using the Illumina TruSeq Stranded mRNAseq Sample Prep kit. The libraries were quantitated by qPCR and sequenced across four lanes on a HiSeq 4000 using a HiSeq 4000 sequencing kit version 1. Fastq files were generated and demultiplexed with the bcl2fastq v2.17.1.14 Conversion Software (IIlumina). Adaptors were trimmed from the 3′ end of the reads and any leading or trailing bases below a quality score of 30 were trimmed via Trimmomatic-0.33, only retaining reads that were 30-bp or longer (Bolger et al. 2014).

The trimmed read files within each subgroup (RR, RS, SR, and SS) were concatenated and assembled using Trinity v2.1.0 (Grabherr et al. 2011). All four resulting assemblies were compared to one another and clustered into groups of transcripts using CD-HIT (Li & Godzik 2006). The longest transcript from each group was used as a representative of that group, generating a final reference transcriptome.

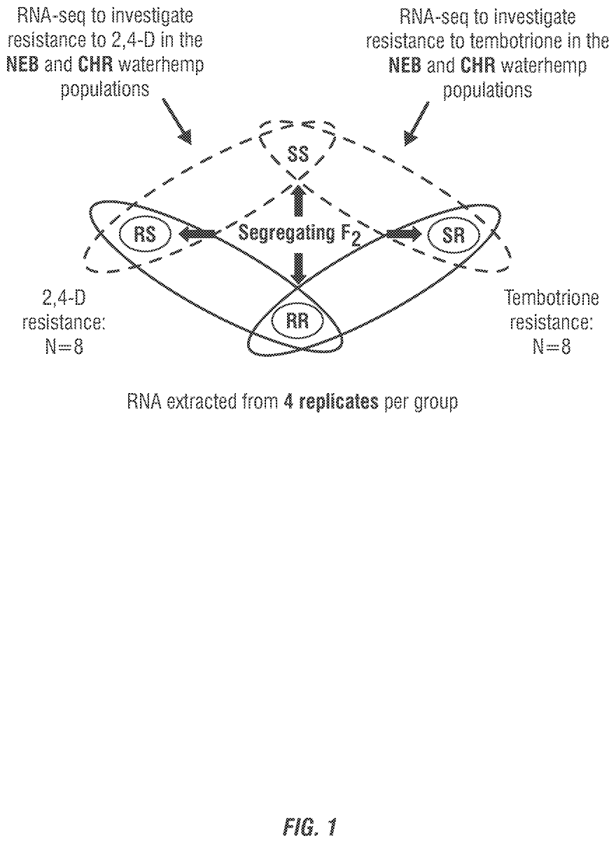

Dose response data from previous work has shown about a 15-fold level of resistance to mesotrione and 9-fold resistance to 2,4-D for the CHR population compared to WUS (Evans et al. 2019). A similar level of 2,4-D resistance has been reported in the NEB population, with 10-fold resistance compared to the Nebraska 2,4-D sensitive population (Bernards et al. 2012) that was reverted to sensitivity by pre-treatment with the cytochrome P450 inhibitor malathion (Figueiredo et al. 2018). For tembotrione, we saw a 43-fold resistance in the CHR population and a 15-fold resistance in the NEB population, compared to WUS (Murphy and Tranel, 2019). In both CHR and NEB populations, resistance to tembotrione and 2,4-D appeared to segregate independently (p-value=0.2457 and 0.1457, respectively). By selecting four F₂ plants with each resistance combination (RR, RS, SR, and SS) we were able to achieve, for each population, eight replicate comparisons for each of two resistant traits from only 16 plants (FIG. 1).

### Example 2: Differential Transcript and Gene Expression Analysis

Each sample was aligned to the reference transcriptome assembly using kallisto (Bray et al. 2016) with the following parameters: -b 100 - -bias - -single - -rf-stranded -l 255 -s 40. These pseudoalignments were then analyzed for differential expression using sleuth (Pimentel et al. 2017) with herbicide sensitivity rating (R vs S) as the condition. The sleuth analysis was carried out for all four comparisons: tembotrione resistant vs sensitive for the NEB population, tembotrione resistant vs sensitive for the CHR population, 2,4-D resistant vs sensitive for the NEB population, and 2,4-D resistant vs sensitive for the CHR population (n=8). Transcripts were further mapped to gene models from a reference genome assembly of A. hypochondriacus (Lightfoot et al. 2017; Genbank accession GCA 000753965.1) to calculate the gene-level differential expression and to anchor genes to scaffolds, potentially identifying any physical clustering of differentially expressed genes (DEGs). GMAP (Wu & Watanabe 2005) was used to align transcripts to the genome in a splice-aware manner (- -cross-species -n 1 - -min-trimmed-coverage=0.80 - -min-identity=0.80). This gene-transcript mapping table was then fed into sleuth, which was rerun in gene mode to calculate differential gene expression between herbicide-resistant and sensitive cohorts. Genes with a Benjamini-Hochberg corrected p-value (Benjamini & Hochberg 1995) of 0.1 or less were considered DEGs and used in further analyses.

The transcriptome assembled into 57,106 transcripts for a total length of 98,112,700 bp. The 32 libraries (16 for each population) were all sequenced to a minimum of 40 million reads per sample (total reads sequenced ranged from 40,800,978 to 54,938,593 bp). Over 80% of reads aligned to the transcriptome for each sample with an average of 81.3% alignment across all libraries, resulting in approximately ˜40× coverage across the entire transcriptome.

For the CHR F₂ population, there were 39 differentially expressed transcripts (DETs) between 2,4-D resistant and 2,4-D sensitive plants and 121 DETs between tembotrione resistant and sensitive plants. In the NEB F₂ population, 1445 transcripts were found to be differentially expressed between 2,4-D resistant and sensitive plants and 115 between tembotrione resistant and sensitive plants.

Of the differentially expressed genes that emerged from the data for all four comparisons, the most likely candidates for herbicide resistance were identified based on their relative rank, fold-change expression, and gene annotation as a possible metabolic resistance gene, as supported by previous publications suggesting an herbicide-metabolism-based resistance mechanism for these populations (Figueiredo et al., 2018; Evans et al., 2019).

Quantitative PCR primers were developed for each candidate gene (TABLE 3). Primers were also created for six housekeeping genes and PCR efficiencies were calculated for all primer sets using a 5-step log-scale serial dilution of cDNA. Only primer sets that showed a PCR efficiency close to 100% (+/−5%) were retained and used for further analyses.

TABLE 3

| Name | Sequence |
|---|---|
| CYP81E8_qF | GTACTTTGATTGAACAGTTGCTGGATTTGC |
| | (SEQ ID NO: 5) |
| CYP81E8_qR | AGGTTCGTGCTGTGGTTTCTGATC |
| | (SEQ ID NO: 6) |
| ABCC10_qF | TGTGGAGAAGTAGGCTCTGGAAAATCA |
| | (SEQ ID NO: 7) |
| ABCC10_qR | TCCGACCATAAACTTCAACAGTGCCT |
| | (SEQ ID NO: 8) |
| CYP71A1_qF | GGTGTTACAATGGATCATATTGGTGTCAAAGC |
| | (SEQ ID NO: 9) |
| CYP71A1_qR | GATGCCTTAATAGTTCTGCCATTGTCCATTC |
| | (SEQ ID NO: 10) |
| CYP72A219_qF | TCAAACCCACTCATAAAGAAGGTCGCAC |
| | (SEQ ID NO: 11) |
| CYP72A219_qR | TCATCTCATTAGAACTTTCCCACATTGCTG |
| | (SEQ ID NO: 12) |
| BTBTOZ_qF | GATCCATATCAATTCAAGGTGTCCCACATG |
| | (SEQ ID NO: 13) |
| BTBTOZ_qR | GGAACAACATACACATGCGACAATACCAG |
| | (SEQ ID NO: 14) |
| CYP97B2_qF | ACCGAAAGCATTTGTCGTTGTCTCG |
| | (SEQ ID NO: 15) |
| CYP97B2_qR | TGGGTTGGAGGATTTCAGCAAGAACT |
| | (SEQ ID NO: 16) |
| ABCI11_qF | TGGACGAAGTGGCAGTGGAAAGA |
| | (SEQ ID NO: 17) |
| ABCI11_qR | ACGCCCATCCTCTCCtTATCCTTGT |
| | (SEQ ID NO: 18) |
| UDPflav_qF | ACCATCATCGTCGGTTGTGTTTCTCT |
| | (SEQ ID NO: 19) |
| UDPflav_qR | TCCTCCAACCCTTTCGCAATCTCTT |
| | (SEQ ID NO: 20) |
| GAPDH_qF | GTGGTGCCAAGAAGGTTGTCATTT |
| | (SEQ ID NO: 21) |
| GAPDH_qR | AGGGAGCAAGGCAGTTGGTG |
| | (SEQ ID NO: 22) |
| EF1alpha_qF | CGTGTGATTGAAAGATTTGAGAAGGAAGC |
| | (SEQ ID NO: 23) |
| EF1alpha_qR | ATACCACGCTCACGCTCTGCT |
| | (SEQ ID NO: 24) |
| 60S-RBP_qF | CTTGTGAGAAGAACTGGTAGCAAA |
| | (SEQ ID NO: 25) |
| 60S-RBP_qR | GTACTTAATCAGCCTAGACAAAGAAAGG |
| | (SEQ ID NO: 26) |

To validate the differential analysis results, a subset of F₂ plants from both CHR- and NEB-derived populations were selected (n=14), including individuals that were and were not used in the RNA-seq. RNA was extracted from all samples using the Trizol method (previously described) and RNA was converted to cDNA using a ProtoScript First Strand cDNA Synthesis Kit (NEB). Quantitative PCR was performed in triplicate on each sample for each primer set by combining 5 μL of iTaq Universal SYBR Green Supermix (Bio-Rad), 0.5 μL forward primer (10 μM), 0.5 μL reverse primer (10 μM), 3 μL of nuclease-free water, and 1 μL of cDNA. Three housekeeping genes were run on each plate for each sample to serve as endogenous controls and assays were conducted 2-3 times to ensure consistent results. Relative expression was calculated using the 2^{-ΔΔCt} method (Livak & Schmittgen 2001), using a sensitive parent (WUS) as the reference sample. These expression values were then regressed against the phenotypic rating values in R (stats v3.6.1) to test for a significant linear relationship for each population.

One of the most significantly differentially expressed transcripts in the CHR population for 2,4-D resistance was a cytochrome P450 (CYP81E8), also identified as an isoflavone 2′-hydroxylase. This same cytochrome P450 was also found to be significantly overexpressed in 2,4-D resistant plants for the NEB population, pointing to a possible shared resistance mechanism between these two populations despite their disparate geographic origins. Quantitative PCR analysis validated overexpression of CYP81E8, finding strong correlations between its expression and phenotypic response to 2,4-D for both populations (TABLE 4). Other putative resistance genes underwent the same qPCR validation process, confirming higher expression of a glucosyltransferase (UDP-glucose flavonoid 3-O-glucosyltransferase) in NEB plants resistant to the HPPD inhibitor. An ABC transporter that emerged as a DET for the CHR population for tembotrione was also confirmed to correlate with resistance, not only for the HPPD inhibitor, but also for 2,4-D resistance in both populations. All genes were also examined for genomic copy number increase using a qPCR-based assay, and no evidence of gene duplication for any of these DETs was found.

TABLE 4
Linear regression of RT-qPCR expression data for each gene
against phenotypic damage ratings for each population (CHR and NEB) and each chemistry (HPPD and
2,4-D). Significant P-values reported; NS, not significant.

| | HPPD | | 2,4-D | |
|---|---|---|---|---|
| Gene | CHR | NEB | CHR | NEB |
| ABCI11 | NS | NS | NS | NS |
| CYP81E8 | NS | NS | 0.021 | 0.008 |
| ABCC10 | NS | 0.036 | 0.034 | 0.018 |
| UDPflav | NS | 0.047 | NS | NS |
| CYP97B2 | NS | NS | NS | NS |
| CYP71A1 | NS | NS | NS | NS |
| CYP72A219 | NS | NS | NS | NS |
| BTBTOZ | NS | NS | NS | NS |

Differential expression was also measured at the gene level to (1) increase the power and remove any confounding information due to minor transcript isoforms and (2) be able to later map the genes to the genome for spatial gene expression profiling. For the CHR population, 90 and 31 differentially expressed genes (DEGs) were obtained for the 2,4-D comparison and tembotrione comparison, respectively. Again, the NEB population gave higher numbers, with 676 DEGs found for the 2,4-D comparison and 268 DEGs found in the tembotrione comparison.

### Example 3: Co-expression Cluster Analysis

Significant clustering of the DEGs was tested using CROC (Pignatelli et al. 2009). CROC searches for clusters using a hypergeometric test that calculates the probability of getting k number of DEGs (out of n total genes) present in a sliding window along each scaffold. A window size of 1 Mbp and an offset size of 500 kbp was used, calling significant clusters only when the adjusted p-value (FDR) was less than 0.05. A sliding window approach was used to visualize clustering along each of the 16 longest scaffolds using R v3.5.1 (R Core Team 2018). Given a window size of 500 kb and a step size of 500 kb, the number of DEGs was counted within each window and plotted using a custom R script.

Additionally, over-representation of DEGs at the whole-chromosome level was tested by totaling up the number of DEGs across each chromosome and comparing them to the expected number of DEGs on that chromosome using Fisher's Exact test in R. Adjusted p-values (p.adjust, method=‘bonferroni’) were calculated.

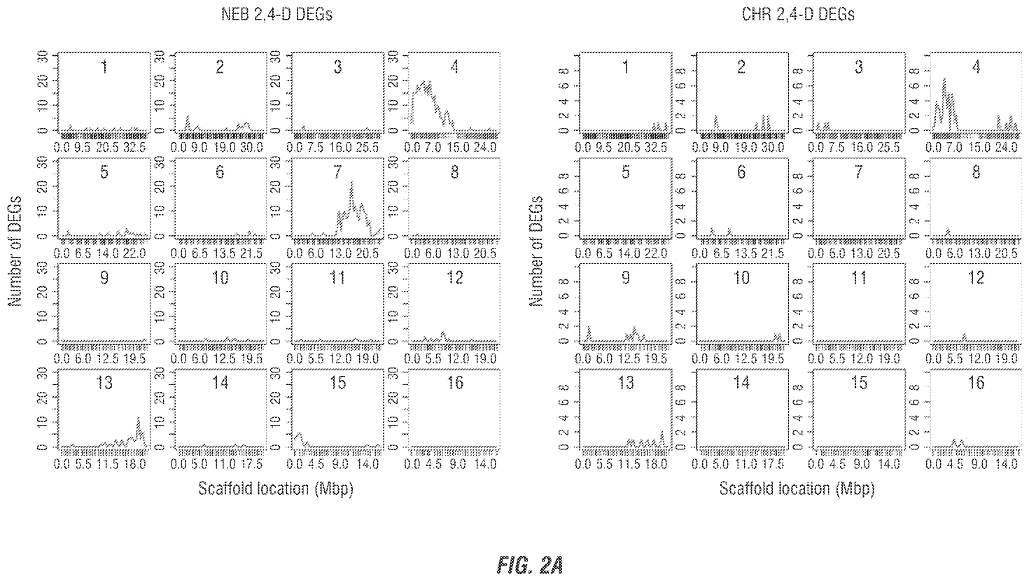

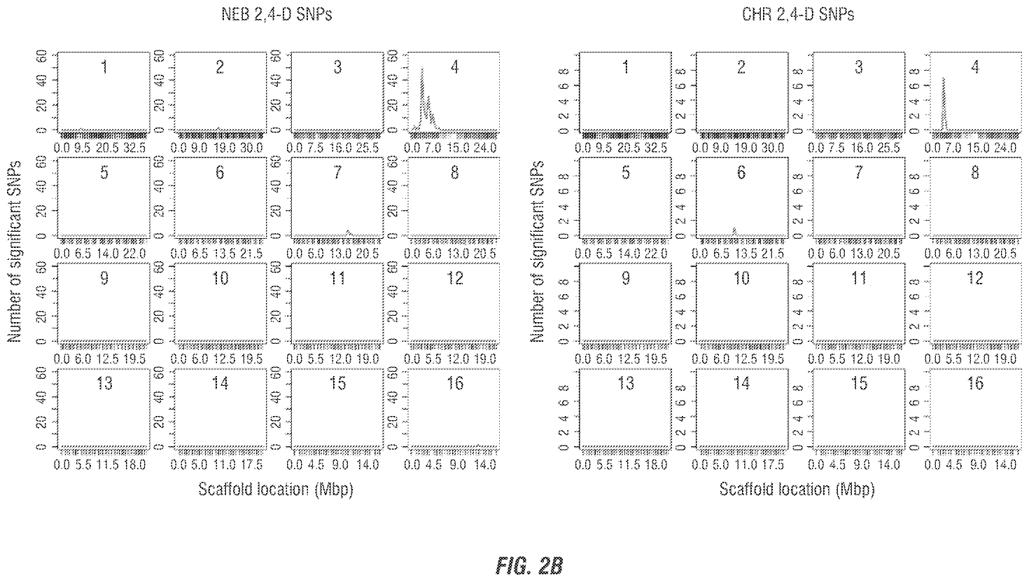

Differentially expressed genes between the 2,4-D resistant and sensitive biotypes of both CHR and NEB were found to physically cluster together in a few chromosomal regions. CROC analyses found significant clustering in a region on scaffold 4 for both populations and a significant region in scaffold 7 for the NEB population (TABLE 5; FIG. 2A). No significant regional clustering was observed for DEGs between HPPD-resistant and -sensitive plants, however, a Fisher's exact test for over-representation of DEGs across the entire chromosome-level scaffolds indicated significantly higher numbers of DEGs than expected on scaffolds 6 and 13 for NEB. This over-representation analysis also identified the significant clustering previously found for the 2,4-D comparisons on scaffold 4 (for CHR and NEB) and scaffold 7 (for NEB) as well as clustering on scaffold 13 for NEB. It may be that the low sample sizes (n=8) were insufficient for adequate resolution of co-expression clusters in the HPPD comparisons.

TABLE 5
Chromosomal cluster testing (using CROC; Pignatelli et al. 2009) of
differentially expressed genes in CHR and NEB for 2,4-D resistance.

| Scaffold | Population | Start | Stop | Adj. P-value |
|---|---|---|---|---|
| Scaffold_4 | CHR | 3469336 | 6412488 | 0.0058 |
| Scaffold_4 | NEB | 3002834 | 9781978 | 1.37E−06 |
| Scaffold_7 | NEB | 14666782 | 16050619 | 0.0021 |

### Example 4: Condition-Specific SNPs

Single nucleotide polymorphisms were called using the best practices outlined by GATK v3.7 (Van der Auwera et al. 2013). Cleaned reads from each RNA-seq sample were first mapped to the A. hypochondriacus genome using STAR v2.5.3 (Dobin et al. 2012) with the following parameters: - -outSAMtype BAM SortedByCoordinate - -quantMode TranscriptomeSAM GeneCounts - -sjdbGTFtagExonParentTranscript Parent. Read groups were assigned and PCR duplicates were removed using Picard Tools v1.95 (The Broad Institute 2019), followed by hard clipping of sequences that extended into the intronic regions using the GATK SplitNCigarReads tool. To correct for any systemic bias in the quality of each aligned base, GATK BaseRecalibrator was run using a set of high-quality SNPs. Since no high-quality SNP datasets exist for A. tuberculatus, a set was created from data generated herein by first running an initial round of variant calling on the uncalibrated data using GATK's HaplotypeCaller and GenotypeGVCFs functions, then hard filtering the SNPs using the following strict parameters: QD<2.0; FS >60.0; MQ<40.0; MQRankSum <−12.5; ReadPosRankSum <−8.0. After base recalibration, variant calling was again run, this time on the calibrated data, using HaplotypeCaller (parameters: -dontUseSoftClippedBases -stand call conf 20.0 - -variant index type LINEAR - -variant index_parameter 128000 -ERC GVCF) and Genotype GVCFs. SNPs were extracted from the final variant file and filtered to include only SNPs that were biallelic and that passed the following parameters: -window 35 -cluster 3 -filter QD<2.0 -filter FS >30.0.

Out of this final SNP dataset, condition-specific SNPs were called using the case/control association analysis in PLINK v1.9 (Chang et al. 2015; SteiB et al. 2012). Due to low sample sizes for each herbicide-resistant versus sensitive comparison (n=8), an adaptive Monte Carlo permutation test with 1000 iterations was also run as part of this association analysis. SNPs that were different between R and S plants with a corrected p-value of 0.05 or less were called as condition-specific SNPs. As with the DEGs, a sliding window approach was used to visualize these condition-specific SNPs, using a window size of 500 kb and a step size of 500 kb.

To check for the presence of any resistant-specific SNPs in these populations, SNPs were called across all genes and condition-specific SNPs (those that varied between resistant and sensitive plants) were identified using Fisher's exact test in PLINK v1.9. Using an adjusted p-value cutoff of 0.05, 10 and 192 SNPs were found to be associated with resistance in the 2,4-D resistant vs sensitive comparison for CHR and NEB, respectively. In both populations, SNPs were found to cluster in the same regions that DEGs were found to cluster. In CHR, 9 out of 10 SNPs were found in the region of scaffold 4 that contained the CYP81E8 gene, while the other SNP was found on scaffold 6. Within the scaffold 4 cluster, there were significant SNPs found in both the CYP81E8 gene as well as a PIN3 auxin efflux carrier gene (which is interesting given 2,4-D is a synthetic auxin). However, 2,4-D resistance cannot be attributed to any of these SNPs since they are in linkage disequilibrium with one another, making it challenging to locate the causal variant. Fine mapping of this region is currently underway. In NEB, 182 SNPs were found in the scaffold 4 region, 6 were found in the scaffold 7 region that also showed a cluster of DEGs in the expression analysis, and the other 4 SNPs were scattered across scaffolds 1, 2, and 16. Sliding window graphs illustrate the clustering of these SNPs, and compared with the DEG sliding window graphs, show the co-occurrence of DEG and SNP clustering (FIG. 2B). No significant SNPs were found between resistant and sensitive plants for the HPPD comparisons. The reason for a lack of SNP clustering in the HPPD comparisons may be due to the more complex nature of this resistance trait, since it has been documented to be a multi-genic trait in these populations (Murphy and Tranel, 2019).

### Example 5: Allele-Specific Expression Analysis

Given the co-occurrence of both differential gene expression and condition-specific SNPs in several regions of the genome, the hypothesis of allele-specific expression was tested using the read count data for each condition-specific SNP to identify all heterozygous individuals (those that showed expression of each allele). Homozygous resistant and sensitive plants at each SNP site were then used to classify each SNP as R or S, then the count data of each R- or S-associated SNP in the heterozygous individuals were used to test for a significant difference in read depth between R and S SNPs using R (rstatix). SNPs and their associated adjusted P-values (Benjamini and Hochberg, p=0.1) were plotted across the scaffold 4 cluster region using R (ggpubr).

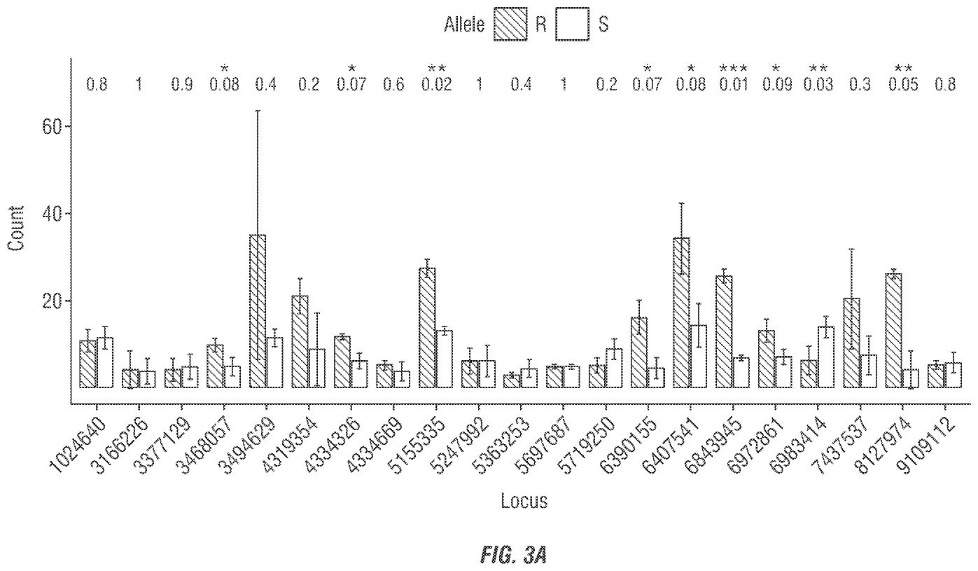

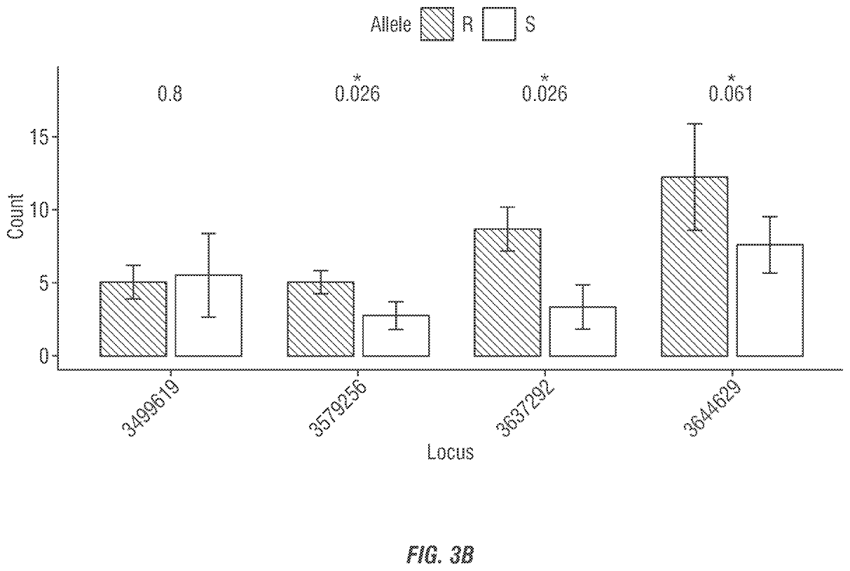

The clustering of condition-specific SNPs with regions of differential gene expression suggested the occurrence of allele-specific expression. Allele-specific expression (ASE) is defined as a form of allelic imbalance, wherein one parental allele is preferentially expressed over another allele (Knight 2004). In the scaffold 4 cluster, nine SNPs were found to be statistically significantly differentially expressed for NEB (FIG. 3A). For all but one, the R allele had significantly higher expression than the S allele, perhaps indicating some cis-acting factor associated with this region, controlling expression. For the CHR population, there were four SNPs that occurred in this scaffold 4 region in heterozygous individuals and three showed significantly different expression between the two alleles (FIG. 3B), again with the R allele showing higher expression than the S allele. ASE may also be occurring in other places along this region, but only the SNPs that were found to occur in a heterozygous state across three or more individuals were included in this analysis.

### Example 6: Cytochrome 81E8 Phylogenetic Analysis

Both the CHR and the NEB population showed the same upregulated allele of a CYP81E8 gene for resistance to 2,4-D, raising the question of whether or not this putative resistance allele evolved independently in each population. Using a previously published (Kreiner et al. 2019) dataset of whole genome sequence from A. tuberculatus samples from Illinois and Canada, a phylogenetic tree was constructed to examine the evolutionary relationship of CYP81E8 from each population. Whole genome or whole transcriptome datasets were aligned to the CDS of CYP81E8 using bowtie2 (Langmead & Salzberg 2012) (parameters: - -no-unal -t -L 20). The sorted bam files were then fed into the same GATK SNP pipeline described above to generate a filtered vcf file. The SNPRelate package in R converted this vcf file to a gds file that could then be used to generate a dendogram based on relatedness (snpgdsHCluster; snpgdsCutTree, n.perm=5000).

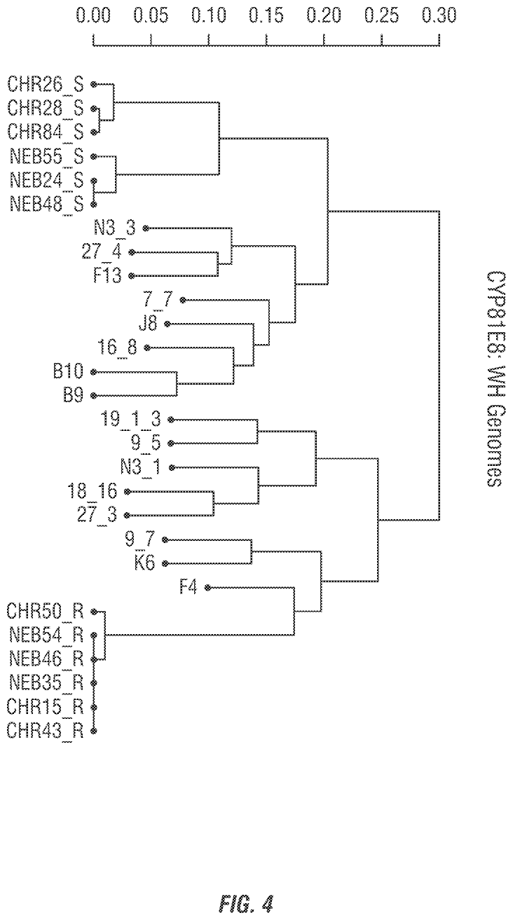

Phylogenetic analysis of the CYP81E8 gene revealed the evolutionary relatedness of each CYP81E8 allele from both the CHR and NEB populations and other A. tuberculatus populations from Illinois, Missouri, and Canada. The CYP81E8 alleles from CHR and NEB separated into three groups representing (1) the 2,4-D sensitive allele from NEB, (2) the 2,4-D sensitive allele from CHR, and (3) the 2,4-D resistant allele in both CHR and NEB (FIG. 4). The separation of the wildtype sensitive alleles from CHR and NEB along with the tight clustering of the 2,4-D resistance-associated CYP81E8 from CHR and NEB provides good evidence that the R allele in both populations has a common evolutionary origin.

### Discussion for Examples 1-6

Strong candidate genes for metabolic-based herbicide resistance were found for 2,4-D in both the CHR and NEB populations in these examples. Both a cytochrome P450 (CYP81E8) and an ABC transporter (ABCC10) showed consistent overexpression in 2,4-D resistant plants compared to 2,4-D sensitive plants. These results support earlier work that found 2,4-D resistance in the NEB population was likely mediated by a cytochrome P450, since the cytochrome P450 inhibitor malathion reversed the resistance phenotype (Figueiredo et al., 2018). The putative resistance allele of this gene co-segregated with additional resistant plants from F₂ populations, and fine mapping is currently underway. Our findings for HPPD-inhibitor resistance, however, were less clear. One candidate gene, a UDP-glucose flavonoid 3-O-glucosyltransferase, was confirmed to be overexpressed in tembotrione-resistant plants compared to tembotrione-sensitive plants. The primary functional annotation of this gene shows it to be involved in fruit ripening, but additional work has shown it to possibly participate in xenobiotic metabolism by glycosylation of exogenous substances (Greisser et al., 2008). The lack of additional candidate HPPD-inhibitor-resistance genes may be due to its multigenic nature (Oliveira et al., 2018), making it difficult to identify the resistance loci. Additionally, our RNA-seq approach focused primarily on identifying genes contributing to resistance via constitutive differential expression, potentially missing other resistance-conferring changes between the plants. A recent RNA-seq study looking into mesotrione resistance in A. tuberculatus did include treated plants and found some evidence of induced expression of cytochrome P450a in resistant plants, compared to sensitive plants (Kohlhase et al., 2019). However, the final list of differentially expressed transcripts in this study was 4800, making the identification of causative resistance genes difficult. Work using a genetic mapping approach to identify HPPD-inhibitor resistance genes in the NEB and CHR populations is currently underway.

Identification of co-expression networks was not extensively pursued in this work due to the fact that plants were not treated with herbicide prior to RNA-seq. Without this shared treatment, it is unlikely that co-expression analysis would yield anything meaningful, since it would measure the random expression differences across the two populations. Indeed, initial forays into co-expression networks yielded no informative results.

In addition to the identification of herbicide resistance gene candidates, this data also reveals some insights into the regulation of herbicide resistance. The physical clustering of DEGs observed for 2,4-D resistance provides evidence for co-expression of co-localized genes, a phenomenon that has been observed in many other species, including yeast (Cohen et al. 2000), Arabidopsis (Williams & Bowles 2004), C. elegans (Chen & Stein 2006), and human (Trinklein et al. 2004). While several of these co-expression clustering examples are found between neighboring gene pairs, co-expression across longer chromosomal intervals has also been reported (Lercher & Hurst 2006; Reimegard et al. 2017). The ability of herbicides to reshape the genomic landscape of weedy species has been recently documented in Ipomoea purpurea, wherein evidence of selective sweeps were found in five genomic regions within glyphosate-resistant populations (Van Etten et al., 2020). Interestingly, enrichment for herbicide detoxification genes was apparent within these regions.

One major implication of this clustering is the likelihood of a shared mechanism of gene regulation for these regions. Regulation of gene expression is a complex process, involving the selective interaction of transcription factors with enhancers, the opening and closing of chromatin to allow/prevent transcription, and the interaction between these two processes (Voss & Hager 2014). We examined the upstream regions of all DEGs and looked for overrepresentation of transcription factor binding sites (TFBSs), but found no evidence of shared enhancer elements. Previous work looking into regulation mechanisms for physically clustered, co-expressed genes has shown that co-expressed gene pairs are often regulated by shared transcription factors, while larger regions of shared expression across 10-20 genes are influenced by a change in the chromatin structure (Batada et al. 2007). However, only a few examples have been studied so far and the interdependent nature of regulatory mechanisms makes it difficult to ascertain direct causes of gene expression. Regardless, more work is needed in these populations to determine the effect of chromatin state on gene expression patterns.

What is claimed is:

1. A modified plant, or a progeny, plant part, or plant cell thereof, having tolerance to an herbicide, the modified plant comprising increased expression of a polynucleotide encoding a cytochrome P450 81E (CYP81E) polypeptide relative to an unmodified plant.

2. The modified plant of claim 1, wherein the modified plant comprises a heterologous polynucleotide encoding the CYP81E polypeptide.

3. The modified plant of claim 1, wherein the CYP81E polypeptide has at least 80%, at least 90%, at least 95%, at least 98%, or at least 99% sequence identity to SEQ ID NO: 2.

4. The modified plant of claim 1, wherein the polynucleotide encoding the CYP81E polypeptide has at least 80%, at least 90%, at least 95%, at least 98%, or at least 99% sequence identity to SEQ ID NO: 1.

5. The modified plant of claim 1, wherein the polynucleotide is operably linked to a heterologous promoter functional in a plant cell.

6. The modified plant of claim 1, wherein the herbicide is an auxin herbicide.

7. The modified plant of claim 6, wherein the auxin herbicide is 2,4-D.

8. The modified plant of claim 1, wherein the plant is dicotyledonous.

9. The modified plant of claim 1, wherein the plant is a crop plant.

10. The modified plant of claim 1, wherein the plant is a soybean, cotton, canola, tobacco, tomato, potato, alfalfa, sugar beet, or sunflower plant.

11. (canceled)

12. A nucleic acid molecule comprising a nucleotide sequence selected from:
(a) a nucleotide sequence encoding a CYP81E polypeptide, wherein the nucleotide sequence has at least 80%, at least 90%, at least 95%, at least 98%, or at least 99% sequence identity to SEQ ID NO: 1; or
(b) a nucleotide sequence encoding a CYP81E polypeptide, wherein the CYP81E polypeptide has at least 80%, at least 90%, at least 95%, at least 98%, or at least 99% sequence identity to SEQ ID NO: 2.

13. The nucleic acid molecule of claim 12, wherein the nucleic acid molecule is an isolated, synthetic, or recombinant nucleic acid molecule.

14. An expression cassette comprising the nucleic acid molecule of claim 12 operably linked to a heterologous promoter functional in a plant cell.

15. A vector comprising the nucleic acid molecule of claim 12.

16. A biological sample comprising the nucleic acid molecule of claim 12.

17. A plant, plant part, or plant cell comprising the nucleic acid molecule of claim 12.

18. A CYP81E polypeptide comprising an amino acid sequence having at least 80%, at least 90%, at least 95%, at least 98%, or at least 99% sequence identity to SEQ ID NO: 2.

19. A method for producing a plant with herbicide tolerance, the method comprising: increasing expression of a polynucleotide encoding a CYP81E polypeptide in the plant, wherein the herbicide tolerance of the plant is increased when compared to a plant that lacks the increased expression.

20. The method of claim 19 comprising introducing to a plant cell a polynucleotide encoding the CYP81E polypeptide, wherein the polynucleotide is operably linked to a heterologous promoter functional in a plant cell; and regenerating a plant from the plant cell.

21. The method of claim 19, wherein the CYP81E polypeptide has at least 80%, at least 90%, at least 95%, at least 98%, or at least 99% sequence identity to SEQ ID NO: 2.

22. The method of claim 19, wherein the polynucleotide encoding the CYP81E polypeptide has at least 80%, at least 90%, at least 95%, at least 98%, or at least 99% sequence identity to SEQ ID NO: 1.

23. The method of claim 19, wherein the herbicide is an auxin herbicide.

24. The method of claim 23, wherein the auxin herbicide is 2,4-D.

25. The method of claim 19, wherein the plant is dicotyledonous.

26. The method of claim 19, wherein the plant is a crop plant.

27. The method of claim 19, wherein the plant a soybean, cotton, canola, tobacco, tomato, potato, alfalfa, sugar beet, or sunflower plant.

28. A method for controlling undesired vegetation at a plant cultivation site, the method comprising:
providing at the site a plant that comprises a polynucleotide encoding a CYP81E polypeptide, wherein expression of the polynucleotide confers to the plant tolerance to an herbicide; and
applying to the site an effective amount of the herbicide.

29. The method of claim 28, wherein the CYP81E polypeptide has at least 80%, at least 90%, at least 95%, at least 98%, or at least 99% sequence identity to SEQ ID NO: 2.

30. The method of claim 28, wherein the polynucleotide encoding the CYP81E polypeptide has at least 80%, at least 90%, at least 95%, at least 98%, or at least 99% sequence identity to SEQ ID NO: 1.

31. The method of claim 28, wherein the polynucleotide is operably linked to a heterologous promoter functional in a plant cell.

32. The method of claim 28, wherein the herbicide is an auxin herbicide.

33. The method of claim 32, wherein the auxin herbicide is 2,4-D.

34. The method of claim 28, wherein the plant is dicotyledonous.

35. The method of claim 28, wherein the plant a soybean, cotton, canola, tobacco, tomato, potato, alfalfa, sugar beet, or sunflower plant.

36. A method for controlling the growth of an herbicide resistant weed at a plant cultivation site, the method comprising:
contacting the weed with a composition comprising a polynucleotide that reduces expression or activity of a CYP81E polypeptide; and
applying to the site an effective amount of the herbicide.

37. The method of claim 36, wherein the polynucleotide is a double-stranded RNA, a single-stranded RNA, or a double-stranded DNA/RNA hybrid polynucleotide.

38. The method of claim 36, wherein the polynucleotide comprises a sequence essentially identical or essentially complementary to at least 18 or more contiguous nucleotides of SEQ ID NO: 1.

39. (canceled)

40. The method of claim 36, wherein the CYP81E polypeptide has at least 80%, at least 90%, at least 95%, at least 98%, or at least 99% sequence identity to SEQ ID NO: 2.

41. The method of claim 36, wherein the herbicide is an auxin herbicide.

42. The method of claim 41, wherein the auxin herbicide is 2,4-D.

43. The method of claim 36, wherein the weed is Amaranthus tuberculatus.

44. The method of claim 36, wherein the composition comprises an agent that enables the polynucleotide to permeate from the surface of the weed into cells of the weed.

45. A product prepared from the plant, plant part, or plant cell of claim 1, wherein the product comprises the polynucleotide encoding the CYP81E polypeptide.

46. The product of claim 45, wherein the product is fodder, seed meal, oil, or seed-treatment-coated seed.

47. A method for producing a plant product, the method comprising processing the plant or plant part of claim 1 to obtain the plant product, wherein the plant product comprises the polynucleotide encoding the CYP81E polypeptide.

48. The method of claim 47, wherein the plant product is fodder, seed meal, oil, or seed-treatment-coated seeds.

49. A method for identifying an herbicide-resistant plant, the method comprising:
providing a biological sample from a plant suspected of having herbicide resistance;
quantifying expression of a CYP81E gene in the biological sample, wherein the CYP81E gene is differentially expressed in an herbicide-resistant plant compared to an herbicide-sensitive plant of the same species; and
determining that the plant is herbicide-resistant based on the quantification.

50. The method of claim 49, wherein the biological sample is from Amaranthus tuberculatus.

51. The method of claim 49, wherein the herbicide is an auxin herbicide.

52. The method of claim 49, wherein the quantifying expression of the CYP81E gene comprises quantifying CYP81E mRNA.

53. The method of claim 49, wherein the quantifying expression of the CYP81E gene comprises quantifying CYP81E polypeptide.

54. The method of claim 49, wherein the CYP81E gene has at least four-fold differential expression in the herbicide-resistant plant compared to the herbicide-sensitive plant prior to application of the herbicide.

55. The method of claim 49, wherein the CYP81E gene has at least 80%, at least 90%, at least 95%, at least 98%, or at least 99% sequence identity to SEQ ID NO: 1.

56. The method of claim 49, wherein the quantifying expression comprises amplifying a nucleic acid using at least two primers.

57. The method of claim 56, wherein the at least two primers comprise SEQ ID NO: 5 and SEQ ID NO: 6.

58. A kit for identifying an herbicide-resistant plant, the kit comprising at least two primers, wherein the at least two primers recognize a CYP81E gene that is differentially expressed in an herbicide-resistant plant compared to an herbicide-sensitive plant of the same species.

59. The kit of claim 58, wherein the wherein the CYP81E gene has at least 80%, at least 90%, at least 95%, at least 98%, or at least 99% sequence identity to SEQ ID NO: 1.

60. The kit of claim 58, further comprising at least one of a positive control and a negative control.

61. The kit of claim 58, further comprising components of a qRT-PCR solution.

62. The kit of claim 58, wherein the plant is Amaranthus tuberculatus and the herbicide is an auxin herbicide.
